(19) United States
(12) Patent Application Publication
(10) Pub. No.: US 2020/0315241 A1
(43) Pub. Date: Oct. 8, 2020

(54) Nicotine Salts and Methods of Making and Using Same

(71) Applicant: Nude Nicotine, Inc., San Diego, CA (US)

(72) Inventor: Jacob Rubenstein, San Diego, CA (US)

(21) Appl. No.: 16/626,831

(22) PCT Filed: Jun. 26, 2018
(86) PCT No.: PCT/US2018/039621

§ 371 Date: Dec. 26, 2019

Related U.S. Application Data
(60) Provisional application No. 62/524,892, filed on Jun. 26, 2017.

(51) Int. Cl. A24B 15/167; A61K 31/455; A61K 9/68; A61K 9/00

(52) U.S. Cl. CPC A24B 15/167; A61K 9/0056; A61K 9/0058; A61K 31/455

(57) ABSTRACT

The present invention provides for compositions including nicotine salts, solutions thereof, methods of manufacture and methods of use. Certain embodiments provide for the delivery of said compositions including by: transdermal, oral, nasal and inhalation modes. Certain embodiments provide nicotine salts and solutions thereof, suitable for or packaged in or with devices including: oral lozenges, chewing gum, transdermal patches, intranasal sprays and intranasal inhalers, e-liquids and e-cigarette or vaping devices.

### CROSS REFERENCE TO RELATED APPLICATIONS

This application is a U.S. national phase application under 35 U.S.C. § 371 claiming priority to International Application No. PCT/US2018/039621 filed Jun. 26, 2018, which claims the benefit of priority from U.S. Provisional Patent Application No. 62/524,892 filed Jun. 26, 2017.

### FIELD OF THE INVENTION

The field generally relates to compositions comprising nicotine salts and methods of making and using same. Specifically, the compounds disclosed herein comprise a nicotine molecule complexed with an acid to form a nicotine salt.

### BACKGROUND OF THE INVENTION

The experience from using combustion type tobacco products, such as cigarettes is preferred by some tobacco users because they describe a perception of a “throat hit” sensation in their respiratory tract. This experience is associated with pleasure for tobacco smokers. In e-cigarettes that use purified, free-base nicotine, this “throat hit” experience does not occur.

Tobacco cigarettes expose users to harmful or potentially harmful constituents (HPHCs), also known as a class of compounds called the Hoffman Analytes. These compounds present an exposure risk to users for diseases such as cancer, emphysema, and/or cardiovascular disease.

Electronic cigarettes (e-cigarettes) which heat a solution of dilute nicotine containing solution (e-Liquid) that was purified from tobacco, might reduce the exposure risk to HPHCs for e-cigarette users compared to combustible tobacco cigarettes because the e-Liquid is vaporized and not combusted which produces more of the HPHCs.

Raw nicotine is commonly extracted from tobacco by adding a base to the tobacco leaf slurry to saponify and partition it in a liquid-liquid extraction system. The raw nicotine may be further purified by column chromatography or distillation to yield high purity free-base nicotine which has a pH of around 8 to 11. Dilute free-base nicotine is commonly used in e-cigarettes.

### SUMMARY OF THE INVENTION

Certain embodiments of the present inventions provide compositions including nicotine salts, solutions thereof, methods of manufacture and methods of use. Certain embodiments provide for the delivery of said compositions including by: transdermal, oral, nasal and inhalation modes. Certain embodiments provide nicotine salts and solutions thereof, suitable for or packaged in or with devices including: oral lozenges, chewing gum, transdermal patches, intranasal sprays and intranasal inhalers, e-liquids and e-cigarette or vaping devices.

### BRIEF DESCRIPTION OF THE DRAWINGS

FIG. 1 is a diagram of a nicotine molecule illustrated as a diprotic base with pKa's of 3.12 (pyridine ring) and 8.02 (pyrrolidine ring).

FIG. 2 is a chart showing the titration curve for nicotine with hydrochloric acid. The lower panel of FIG. 2 is an illustration of the nicotine molecule at pH's corresponding to acid, neutral and basic conditions and these corresponded to the positions in the chart.

FIG. 3 is a drawing of a bridged nicotine salt complex. The nicotine molecule is shown having a malate bridge including hydrogen bonding to both the pyridine nitrogen and the pyrrolidine nitrogen.

FIG. 4A shows a nicotine levulinic acid complex. FIG. 4B illustrates a heterogenous salt mixture (nicotine N-benzoate-N′-malate). FIG. 4C shows a novel homogenous salt.

### DETAILED DESCRIPTION OF THE INVENTION

Certain embodiments of the present invention provide compositions of nicotine salt complexes and solutions containing said complexes. Embodiments include nicotine salt complexes having nicotine molecules associated with one or more organic acids, or conjugate bases thereof, which is the deprotonated form of its respective organic acid, the conjugate base of the an organic acid is also referred to as the weak base form of the organic acids. In certain embodiments, an organic acid bonds with the nicotine through hydrogen bonding, although the present invention is not limited by mechanism.

In certain embodiments, the first, or preferential, bonding occurs at the N-methyl pyrrolidinyl nitrogen due to its higher basicity (larger pKa, or dissociation constant). In certain embodiments, this dissociation constant (pKa) value for the N-methyl pyrrolidinyl nitrogen of nicotine is approximately eight; thus, when nicotine molecules (or solution therein) have a pH of approximately eight, then fifty percent of them are protonated at the N-methyl pyrrolidinyl nitrogen and fifty percent are not. In certain embodiments, when the pH of the nicotine is approximately at pH seven, then about ninety percent are protonated at the N-methyl pyrrolidinyl nitrogen and ten percent are not; and accordingly, when nicotine molecules are approximately at pH six, ninety nine percent are protonated at the N-methyl pyrrolidinyl nitrogen and one percent are not.

Certain embodiments herein provide nicotine salt complexes, wherein the nicotine molecule's two nitrogen centers (in their respective pyrrolidine and pyridine rings) associate with or conjugate to different organic acids (or the conjugate bases thereof). Embodiments herein refer to a nicotine molecule having different organic acid constituents as “higher order” nicotine salt complexes or “heterogenous nicotine salt complexes.” In certain embodiments, more than two organic acids are paired with nicotine molecules providing a variety of embodiments of heterogenous nicotine salt complexes as the different organic acids associate with different nicotine molecules as permitted by increasing the number of organic acid molecules. Various embodiments herein provide heterogenous nicotine salt complexes including two, three, four, five, six, seven, eight, nine, ten, twelve, fifteen, or twenty or more organic acids.

Certain embodiments of the present invention provide a heterogenous nicotine salt complex, comprising a nicotine molecule having a first organic acid associated with the nitrogen of the N-methyl pyrrolidine and a second organic acid associated with the nitrogen of the pyridine ring, wherein the first and second organic acids are different (i.e., not identical).

Certain embodiments provide solutions, comprising: one or more heterogenous nicotine salt complexes. In certain embodiments, a solution is provided, comprising: more than two organic acids and nicotine, wherein various nicotine molecules include two different organic acids and there is, or optionally can be, a variety of nicotine salt complexes present in the solution.

In certain embodiments, organic acids having fewer than 6 carbons in a straight (unbranched) chain (AKA caproate/hexanoate) can form 1:1 single complexes or 1:2 higher order salt complexes. Certain exemplary embodiments include: nicotine dicitrate, nicotine dibenzoate, nicotine ditartrate, and nicotine dioxalate.

In certain embodiments, branched molecules have a separation of binding sites for heterogenous nicotine salt complexes allowing for 1:2 formations (nicotine:organic acid, molar ratios), for example nicotine dicitrate.

In certain embodiments, steric hinderance limits the range of organic acids that are able to bond with nicotine to form a heterogeneous complex. For example, in certain embodiments, a first organic acid bonds with the pyrrolidine nitrogen center and steric hinderance limits the types of organic acids able to bond with the pyridine nitrogen center. In certain embodiments, the steric hinderance is increased with an increase in size of the first organic acid, an increase in the electronegativity of the first organic acid or both.

Certain embodiments provide a method of selecting a second organic acid for bonding with the pyridine nitrogen center in view of the nicotine having a first organic acid that is bonded with the pyrrolidine nitrogen center, comprising: identifying an organic acid having a size smaller than the first organic acid, an electronegative character that is less, or both. The organic acids listed in Table 1, column B, for example, are suitable as the second organic acid given the selection of the first organic acid in shown in column A, in preferred embodiments of heterogeneous nicotine salts.

TABLE 1
Embodiments of the Preferred Second Organic Acids (in Column B) for
Pairing with the Selected First Organic Acid in Column A for reaction
with Nicotine or for Forming a Heterogeneous Nicotine Salt Complex

| Column A | |
|---|---|
| Showing a Selected | |
| Organic Acid | Column B |
| Bonded at the | Preferred Organic Acid(s) for Bonded at the Pyridine Ring, |
| Pyrrolidine Ring | in View of the Selection of Organic Acid in Column A |
| 2- | Malonic Acid, Tartaric Acid, Glutaric Acid, Hexanoic Acid, Malic |
| Hydroxyisocaproic | Acid, Propionic Acid, Glutamic Acid, Oxalic Acid, Salicylic Acid, Lactic |
| Acid | Acid, Propiolic Acid, Acetic Acid, Valeric Acid, Acetoacetic acid, Pyruvic |
| | Acid, Glycolic Acid, Formic Acid, Succinic Acid, Levuliniuc Acid, Adipitic |
| | Acid, 2-Hydroxyisocaproic Acid, Phthalic Acid, Cinnamic Acid, Acetylsalicylic |
| | Acid, Citric Acid, Isocitric Acid, Fumaric Acid, Gluconic Acid, Gallic |
| | Acid, Benzoic Acid, 4-hydroxybenxoic Acid, Trimesic Acid, Nicotinic |
| | Acid, Cis-Aconitic Acid, Trans-Aconitic Acid, Cinnamic Acid, Vanillic |
| | acid, Aspartic Acid, oxaloacetic acid, mesooxalic acid, alpha-keto-glutaric |
| | acid, beta-keto-glutaric acid, hydroxypyruvic acid, 3-mercaptopyruvic acid |
| 3- | Malonic Acid, Tartaric Acid, Glutaric Acid, Hexanoic Acid, Malic |
| mercaptopyruvic | Acid, Propionic Acid, Glutamic Acid, Oxalic Acid, Salicylic Acid, Lactic |
| acid | Acid, Propiolic Acid, Acetic Acid, Valeric Acid, Acetoacetic acid, Pyruvic |
| | Acid, Glycolic Acid, Formic Acid, Succinic Acid, Levuliniuc Acid, Adipitic |
| | Acid, 2-Hydroxyisocaproic Acid, Phthalic Acid, Cinnamic Acid, Acetylsalicylic |
| | Acid, Citric Acid, Isocitric Acid, Fumaric Acid, Gluconic Acid, Gallic |
| | Acid, Benzoic Acid, 4-hydroxybenxoic Acid, Trimesic Acid, Nicotinic |
| | Acid, Cis-Aconitic Acid, Trans-Aconitic Acid, Cinnamic Acid, Vanillic |
| | acid, oxaloacetic acid, mesooxalic acid, alpha-keto-glutaric acid, beta-keto- |
| | glutaric acid, hydroxypyruvic acid, 3-mercaptopyruvic acid, Aspartic Acid |
| 4- | Malonic Acid, Tartaric Acid, Glutaric Acid, Hexanoic Acid, Malic |
| hydroxybenxoic | Acid, Propionic Acid, Glutamic Acid, Oxalic Acid, Salicylic Acid, Lactic |
| Acid | Acid, Propiolic Acid, Acetic Acid, Valeric Acid, Acetoacetic acid, Pyruvic |
| | Acid, Glycolic Acid, Formic Acid, Succinic Acid, Levuliniuc Acid, Adipitic |
| | Acid, 2-Hydroxyisocaproic Acid, Phthalic Acid, Cinnamic Acid, Acetylsalicylic |
| | Acid, Citric Acid, Isocitric Acid, Fumaric Acid, Gluconic Acid, Gallic |
| | Acid, Benzoic Acid, 4-hydroxybenxoic Acid, Trimesic Acid, Nicotinic |
| | Acid, Cis-Aconitic Acid, Trans-Aconitic Acid, Cinnamic Acid, Vanillic |
| | acid, oxaloacetic acid, mesooxalic acid, alpha-keto-glutaric acid, beta-keto- |
| | glutaric acid, hydroxypyruvic acid, 3-mercaptopyruvic acid, Aspartic Acid |
| 4-hydroxy-2- | Malonic Acid, Tartaric Acid, Glutaric Acid, Hexanoic Acid, Malic |
| oxopentanoic acid | Acid, Propionic Acid, Glutamic Acid, Oxalic Acid, Lactic Acid, Propiolic |
| | Acid, Acetic Acid, Valeric Acid, Acetoacetic acid, Pyruvic Acid, Glycolic |
| | Acid, Formic Acid, Succinic Acid, Levulinic Acid, Adipitic Acid, 2- |
| | Hydroxyisocaproic Acid, Citric Acid, Isocitric Acid, Fumaric |
| | Acid, Acetylsalicylic Acid, Phthalic Acid, Cinnamic Acid, Gluconic Acid, Gallic |
| | Acid, Benzoic Acid, 4-hydroxybenxoic Acid, Trimesic Acid, Nicotinic |
| | Acid, Cis-Aconitic Acid, Trans-Aconitic Acid, Cinnamic Acid, Vanillic |
| | acid, oxaloacetic acid, mesooxalic acid, alpha-keto-glutaric acid, beta-keto- |
| | glutaric acid, hydroxypyruvic acid, 3-mercaptopyruvic acid, Aspartic Acid |
| 4- | Malonic Acid, Tartaric Acid, Glutaric Acid, Hexanoic Acid, Malic |
| hydroxyphenylpyruvic | Acid, Propionic Acid, Glutamic Acid, Oxalic Acid, Salicylic Acid, Lactic |
| acid | Acid, Propiolic Acid, Acetic Acid, Valeric Acid, Acetoacetic acid, Pyruvic |
| | Acid, Glycolic Acid, Formic Acid, Succinic Acid, Levulinic Acid, Adipitic |
| | Acid, 2-Hydroxyisocaproic Acid, Phthalic Acid, Cinnamic Acid, Citric |
| | Acid, Isocitric Acid, Fumaric Acid, Gluconic Acid, Gallic Acid, Benzoic Acid, 4- |
| | hydroxybenxoic Acid, Trimesic Acid, Nicotinic Acid, Cis-Aconitic Acid, Trans- |
| | Aconitic Acid, Cinnamic Acid, Vanillic acid, oxaloacetic acid, mesooxalic |
| | acid, alpha-keto-glutaric acid, beta-keto-glutaric acid, hydroxypyruvic acid, 3- |
| | mercaptopyruvic acid, Aspartic Acid |
| Acetic Acid | Malonic Acid, Tartaric Acid, Glutaric Acid, Hexanoic Acid, Malic |
| | Acid, Propionic Acid, Glutamic Acid, Oxalic Acid, Salicylic Acid, Lactic |
| | Acid, Propiolic Acid, Acetic Acid, Valeric Acid, Acetoacetic acid, Pyruvic |
| | Acid, Glycolic Acid, Formic Acid, Succinic Acid, Levuliniuc Acid, Adipitic |
| | Acid, 2-Hydroxyisocaproic Acid, Phthalic Acid, Cinnamic Acid, Acetylsalicylic |
| | Acid, Citric Acid, Isocitric Acid, Fumaric Acid, Gluconic Acid, Gallic |
| | Acid, Benzoic Acid, 4-hydroxybenxoic Acid, Trimesic Acid, Nicotinic |
| | Acid, Cis-Aconitic Acid, Trans-Aconitic Acid, Cinnamic Acid, Vanillic |
| | acid, oxaloacetic acid, mesooxalic acid, alpha-keto-glutaric acid, beta-keto- |
| | glutaric acid, hydroxypyruvic acid, 3-mercaptopyruvic acid, Aspartic Acid |
| Acetoacetic acid | Malonic Acid, Tartaric Acid, Glutaric Acid, Hexanoic Acid, Malic |
| | Acid, Propionic Acid, Glutamic Acid, Oxalic Acid, Salicylic Acid, Lactic |
| | Acid, Propiolic Acid, Acetic Acid, Valeric Acid, Acetoacetic acid, Pyruvic |
| | Acid, Glycolic Acid, Formic Acid, Succinic Acid, Levuliniuc Acid, Adipitic |
| | Acid, 2-Hydroxyisocaproic Acid, Phthalic Acid, Cinnamic Acid, Acetylsalicylic |
| | Acid, Citric Acid, Isocitric Acid, Fumaric Acid, Gluconic Acid, Gallic |
| | Acid, Benzoic Acid, 4-hydroxybenxoic Acid, Trimesic Acid, Nicotinic |
| | Acid, Cis-Aconitic Acid, Trans-Aconitic Acid, Cinnamic Acid, Vanillic |
| | acid, oxaloacetic acid, mesooxalic acid, alpha-keto-glutaric acid, beta-keto- |
| | glutaric acid, hydroxypyruvic acid, 3-mercaptopyruvic acid, Aspartic Acid |
| Acetylsalicylic Acid | Malonic Acid, Tartaric Acid, Glutaric Acid, Hexanoic Acid, Malic |
| | Acid, Propionic Acid, Glutamic Acid, Oxalic Acid, Salicylic Acid, Lactic |
| | Acid, Propiolic Acid, Acetic Acid, Valeric Acid, Acetoacetic acid, Pyruvic |
| | Acid, Glycolic Acid, Formic Acid, Succinic Acid, Levuliniuc Acid, Adipitic |
| | Acid, 2-Hydroxyisocaproic Acid, Phthalic Acid, Cinnamic Acid, Acetylsalicylic |
| | Acid, Citric Acid, Isocitric Acid, Fumaric Acid, Gluconic Acid, Gallic |
| | Acid, Benzoic Acid, 4-hydroxybenxoic Acid, Trimesic Acid, Nicotinic |
| | Acid, Cis-Aconitic Acid, Trans-Aconitic Acid, Cinnamic Acid, Vanillic |
| | acid, oxaloacetic acid, mesooxalic acid, alpha-keto-glutaric acid, beta-keto- |
| | glutaric acid, hydroxypyruvic acid, 3-mercaptopyruvic acid, Aspartic Acid |
| Adipitic Acid | Malonic Acid, Tartaric Acid, Glutaric Acid, Hexanoic Acid, Malic |
| | Acid, Propionic Acid, Glutamic Acid, Oxalic Acid, Lactic Acid, Propiolic |
| | Acid, Acetic Acid, Valeric Acid, Acetoacetic acid, Pyruvic Acid, Glycolic |
| | Acid, Formic Acid, Succinic Acid, Levuliniuc Acid, Adipitic Acid, 2- |
| | Hydroxyisocaproic Acid, Phthalic Acid, Cinnamic Acid, Acetylsalicylic |
| | Acid, Citric Acid, Isocitric Acid, Fumaric Acid, Gluconic Acid, Gallic |
| | Acid, Benzoic Acid, 4-hydroxybenxoic Acid, Trimesic Acid, Nicotinic |
| | Acid, Cis-Aconitic Acid, Trans-Aconitic Acid, Cinnamic Acid, Vanillic |
| | acid, Salicylic Acid, oxaloacetic acid, mesooxalic acid, alpha-keto-glutaric |
| | acid, beta-keto-glutaric acid, hydroxypyruvic acid, 3-mercaptopyruvic |
| | acid, Aspartic Acid |
| Alanine | Malonic Acid, Tartaric Acid, Glutaric Acid, Hexanoic Acid, Malic |
| | Acid, Propionic Acid, Glutamic Acid, Oxalic Acid, Salicylic Acid, Lactic |
| | Acid, Propiolic Acid, Acetic Acid, Valeric Acid, Acetoacetic acid, Pyruvic |
| | Acid, Glycolic Acid, Formic Acid, Succinic Acid, Levulinic Acid, Adipitic |
| | Acid, 2-Hydroxyisocaproic Acid, Phthalic Acid, Cinnamic Acid, Acetylsalicylic |
| | Acid, Citric Acid, Isocitric Acid, Fumaric Acid, Gluconic Acid, Gallic |
| | Acid, Benzoic Acid, 4-hydroxybenxoic Acid, Trimesic Acid, Nicotinic |
| | Acid, Cis-Aconitic Acid, Trans-Aconitic Acid, Cinnamic Acid, Vanillic |
| | acid, Aspartic Acid, Glutamic |
| | Acid, Valine, Leucine, Isoleucine, Alanine, Arginine, Lysine, Proline, Cysteine, |
| | Threonine, Methionine, Histidine, Phenylalanine, Tyrosine, Tryptophan, Asparagine, |
| | Glycine, Serine, oxaloacetic acid, mesooxalic acid, alpha-keto-glutaric |
| | acid, beta-keto-glutaric acid, hydroxypyruvic acid, 3-mercaptopyruvic |
| | acid, Aspartic Acid |
| Arginine | Malonic Acid, Tartaric Acid, Glutaric Acid, Hexanoic Acid, Malic |
| | Acid, Propionic Acid, Glutamic Acid, Oxalic Acid, Salicylic Acid, Lactic |
| | Acid, Propiolic Acid, Acetic Acid, Valeric Acid, Acetoacetic acid, Pyruvic |
| | Acid, Glycolic Acid, Formic Acid, Succinic Acid, Levulinic Acid, Adipitic |
| | Acid, 2-Hydroxyisocaproic Acid, Phthalic Acid, Cinnamic Acid, Acetylsalicylic |
| | Acid, Citric Acid, Isocitric Acid, Fumaric Acid, Gluconic Acid, Gallic |
| | Acid, Benzoic Acid, 4-hydroxybenxoic Acid, Trimesic Acid, Nicotinic |
| | Acid, Cis-Aconitic Acid, Trans-Aconitic Acid, Cinnamic Acid, Vanillic |
| | acid, Aspartic Acid, Glutamic |
| | Acid, Valine, Leucine, Isoleucine, Alanine, Arginine, Lysine, Proline, Cysteine, |
| | Threonine, Methionine, Histidine, Phenylalanine, Tyrosine, Tryptophan, Asparagine, |
| | Glycine, Serine, oxaloacetic acid, mesooxalic acid, alpha-keto-glutaric |
| | acid, beta-keto-glutaric acid, hydroxypyruvic acid, 3-mercaptopyruvic |
| | acid, Aspartic Acid |
| Asparginine | Malonic Acid, Tartaric Acid, Glutaric Acid, Hexanoic Acid, Malic |
| | Acid, Propionic Acid, Glutamic Acid, Oxalic Acid, Salicylic Acid, Lactic |
| | Acid, Propiolic Acid, Acetic Acid, Valeric Acid, Acetoacetic acid, Pyruvic |
| | Acid, Glycolic Acid, Formic Acid, Succinic Acid, Levulinic Acid, Adipitic |
| | Acid, 2-Hydroxyisocaproic Acid, Phthalic Acid, Cinnamic Acid, Acetylsalicylic |
| | Acid, Citric Acid, Isocitric Acid, Fumaric Acid, Gluconic Acid, Gallic |
| | Acid, Benzoic Acid, 4-hydroxybenxoic Acid, Trimesic Acid, Nicotinic |
| | Acid, Cis-Aconitic Acid, Trans-Aconitic Acid, Cinnamic Acid, Vanillic |
| | acid, Aspartic Acid, Glutamic |
| | Acid, Valine, Leucine, Isoleucine, Alanine, Arginine, Lysine, Proline, Cysteine, |
| | Threonine, Methionine, Histidine, Phenylalanine, Tyrosine, Tryptophan, Asparagine, |
| | Glycine, Serine, oxaloacetic acid, mesooxalic acid, alpha-keto-glutaric |
| | acid, beta-keto-glutaric acidhydroxypyruvic acid, 3-mercaptopyruvic |
| | acid, Aspartic Acid |
| Aspartic Acid | Malonic Acid, Tartaric Acid, Glutaric Acid, Hexanoic Acid, Malic |
| | Acid, Propionic Acid, Glutamic Acid, Oxalic Acid, Salicylic Acid, Lactic |
| | Acid, Propiolic Acid, Acetic Acid, Valeric Acid, Acetoacetic acid, Pyruvic |
| | Acid, Glycolic Acid, Formic Acid, Succinic Acid, Levulinic Acid, Adipitic |
| | Acid, 2-Hydroxyisocaproic Acid, Phthalic Acid, Cinnamic Acid, Acetylsalicylic |
| | Acid, Citric Acid, Isocitric Acid, Fumaric Acid, Gluconic Acid, Gallic |
| | Acid, Benzoic Acid, 4-hydroxybenxoic Acid, Trimesic Acid, Nicotinic |
| | Acid, Cis-Aconitic Acid, Trans-Aconitic Acid, Cinnamic Acid, Vanillic |
| | acid, Aspartic Acid, Glutamic |
| | Acid, Valine, Leucine, Isoleucine, Alanine, Arginine, Lysine, Proline, Cysteine, |
| | Threonine, Methionine, Histidine, Phenylalanine, Tyrosine, Tryptophan, Asparagine, |
| | Glycine, Serine, oxaloacetic acid, mesooxalic acid, alpha-keto-glutaric |
| | acid, beta-keto-glutaric acid, hydroxypyruvic acid, 3-mercaptopyruvic |
| | acid, Aspartic Acid |
| Benzoic Acid | Malonic Acid, Tartaric Acid, Glutaric Acid, Hexanoic Acid, Malic |
| | Acid, Propionic Acid, Glutamic Acid, Oxalic Acid, Salicylic Acid, Lactic |
| | Acid, Propiolic Acid, Acetic Acid, Valeric Acid, Acetoacetic acid, Pyruvic |
| | Acid, Glycolic Acid, Formic Acid, Succinic Acid, Levuliniuc Acid, Adipitic |
| | Acid, 2-Hydroxyisocaproic Acid, Phthalic Acid, Cinnamic Acid, Acetylsalicylic |
| | Acid, Citric Acid, Isocitric Acid, Fumaric Acid, Gluconic Acid, Gallic |
| | Acid, Benzoic Acid, 4-hydroxybenxoic Acid, Trimesic Acid, Nicotinic |
| | Acid, Cis-Aconitic Acid, Trans-Aconitic Acid, Cinnamic Acid, Vanillic |
| | acid, oxaloacetic acid, mesooxalic acid, alpha-keto-glutaric acid, beta-keto- |
| | glutaric acid, hydroxypyruvic acid, 3-mercaptopyruvic acid, Aspartic Acid |
| Cinnamic Acid | Malonic Acid, Tartaric Acid, Glutaric Acid, Hexanoic Acid, Malic |
| | Acid, Propionic Acid, Glutamic Acid, Oxalic Acid, Lactic Acid, Propiolic |
| | Acid, Acetic Acid, Valeric Acid, Acetoacetic acid, Pyruvic Acid, Glycolic |
| | Acid, Formic Acid, Succinic Acid, Levuliniuc Acid, Adipitic Acid, 2- |
| | Hydroxyisocaproic Acid, Citric Acid, Isocitric Acid, Fumaric Acid, Cinnamic |
| | acid, oxaloacetic acid, mesooxalic acid, alpha-keto-glutaric acid, beta-keto- |
| | glutaric acid, hydroxypyruvic acid, 3-mercaptopyruvic acid, Aspartic Acid |
| Cis-Aconitic Acid | Malonic Acid, Tartaric Acid, Glutaric Acid, Hexanoic Acid, Malic |
| | Acid, Propionic Acid, Glutamic Acid, Oxalic Acid, Lactic Acid, Propiolic |
| | Acid, Acetic Acid, Valeric Acid, Acetoacetic acid, Pyruvic Acid, Glycolic |
| | Acid, Formic Acid, Succinic Acid, Levuliniuc Acid, Adipitic Acid, 2- |
| | Hydroxyisocaproic Acid, Citric Acid, Isocitric Acid, Fumaric Acid, oxaloacetic |
| | acid, mesooxalic acid, alpha-keto-glutaric acid, beta-keto-glutaric |
| | acid, hydroxypyruvic acid, Cis-Aconitic Acid, 3-mercaptopyruvic acid, Aspartic |
| | Acid |
| Citric Acid | Malonic Acid, Tartaric Acid, Glutaric Acid, Hexanoic Acid, Malic |
| | Acid, Propionic Acid, Glutamic Acid, Oxalic Acid, Lactic Acid, Propiolic |
| | Acid, Acetic Acid, Valeric Acid, Acetoacetic acid, Pyruvic Acid, Glycolic |
| | Acid, Formic Acid, Succinic Acid, Levuliniuc Acid, Adipitic Acid, 2- |
| | Hydroxyisocaproic Acid, Phthalic Acid, Cinnamic Acid, Acetylsalicylic |
| | Acid, Citric Acid, Isocitric Acid, Fumaric Acid, Gluconic Acid, Gallic |
| | Acid, Benzoic Acid, 4-hydroxybenxoic Acid, Trimesic Acid, Nicotinic |
| | Acid, Cis-Aconitic Acid, Trans-Aconitic Acid, Cinnamic Acid, Vanillic |
| | acid, Salicylic Acid, oxaloacetic acid, mesooxalic acid, alpha-keto-glutaric |
| | acid, beta-keto-glutaric acid, hydroxypyruvic acid, 3-mercaptopyruvic |
| | acid, Aspartic Acid |
| Cysteine | Malonic Acid, Tartaric Acid, Glutaric Acid, Hexanoic Acid, Malic |
| | Acid, Propionic Acid, Glutamic Acid, Oxalic Acid, Salicylic Acid, Lactic |
| | Acid, Propiolic Acid, Acetic Acid, Valeric Acid, Acetoacetic acid, Pyruvic |
| | Acid, Glycolic Acid, Formic Acid, Succinic Acid, Levulinic Acid, Adipitic |
| | Acid, 2-Hydroxyisocaproic Acid, Phthalic Acid, Cinnamic Acid, Acetylsalicylic |
| | Acid, Citric Acid, Isocitric Acid, Fumaric Acid, Gluconic Acid, Gallic |
| | Acid, Benzoic Acid, 4-hydroxybenxoic Acid, Trimesic Acid, Nicotinic |
| | Acid, Cis-Aconitic Acid, Trans-Aconitic Acid, Cinnamic Acid, Vanillic |
| | acid, Aspartic Acid, Glutamic |
| | Acid, Valine, Leucine, Isoleucine, Alanine, Arginine, Lysine, Proline, Cysteine, |
| | Threonine, Methionine, Histidine, Phenylalanine, Tyrosine, Tryptophan, Asparagine, |
| | Glycine, Serine, oxaloacetic acid, mesooxalic acid, alpha-keto-glutaric |
| | acid, beta-keto-glutaric acid, hydroxypyruvic acid, 3-mercaptopyruvic |
| | acid, Aspartic Acid |
| Formic Acid | Malonic Acid, Tartaric Acid, Glutaric Acid, Hexanoic Acid, Malic |
| | Acid, Propionic Acid, Glutamic Acid, Oxalic Acid, Lactic Acid, Propiolic |
| | Acid, Acetic Acid, Valeric Acid, Acetoacetic acid, Pyruvic Acid, Glycolic |
| | Acid, Formic Acid, Succinic Acid, Levuliniuc Acid, Adipitic Acid, 2- |
| | Hydroxyisocaproic Acid, Phthalic Acid, Cinnamic Acid, Acetylsalicylic |
| | Acid, Citric Acid, Isocitric Acid, Fumaric Acid, Gluconic Acid, Gallic |
| | Acid, Benzoic Acid, 4-hydroxybenxoic Acid, Trimesic Acid, Nicotinic |
| | Acid, Cis-Aconitic Acid, Trans-Aconitic Acid, Cinnamic Acid, Vanillic |
| | acid, Salicylic Acid, oxaloacetic acid, mesooxalic acid, alpha-keto-glutaric |
| | acid, beta-keto-glutaric acid, hydroxypyruvic acid, 3-mercaptopyruvic |
| | acid, Aspartic Acid |
| Fumaric Acid | Malonic Acid, Tartaric Acid, Glutaric Acid, Hexanoic Acid, Malic |
| | Acid, Propionic Acid, Glutamic Acid, Oxalic Acid, Lactic Acid, Propiolic |
| | Acid, Acetic Acid, Valeric Acid, Acetoacetic acid, Pyruvic Acid, Glycolic |
| | Acid, Formic Acid, Succinic Acid, Levuliniuc Acid, Adipitic Acid, 2- |
| | Hydroxyisocaproic Acid, Phthalic Acid, Cinnamic Acid, Acetylsalicylic |
| | Acid, Citric Acid, Isocitric Acid, Fumaric Acid, Gluconic Acid, Gallic |
| | Acid, Benzoic Acid, 4-hydroxybenxoic Acid, Trimesic Acid, Nicotinic |
| | Acid, Cis-Aconitic Acid, Trans-Aconitic Acid, Cinnamic Acid, Vanillic |
| | acid, Salicylic Acid, oxaloacetic acid, mesooxalic acid, alpha-keto-glutaric |
| | acid, beta-keto-glutaric acid, hydroxypyruvic acid, 3-mercaptopyruvic |
| | acid, Aspartic Acid |
| Gallic Acid | Malonic Acid, Tartaric Acid, Glutaric Acid, Hexanoic Acid, Malic |
| | Acid, Propionic Acid, Glutamic Acid, Oxalic Acid, Lactic Acid, Propiolic |
| | Acid, Acetic Acid, Valeric Acid, Acetoacetic acid, Pyruvic Acid, Glycolic |
| | Acid, Gallic Acid, Formic Acid, Succinic Acid, Levuliniuc Acid, Adipitic |
| | Acid, 2-Hydroxyisocaproic Acid, Citric Acid, Isocitric Acid, Fumaric |
| | Acid, oxaloacetic acid, mesooxalic acid, alpha-keto-glutaric acid, beta-keto- |
| | glutaric acid, hydroxypyruvic acid, 3-mercaptopyruvic acid, Aspartic Acid |
| Gluconic Acid | Malonic Acid, Tartaric Acid, Glutaric Acid, Hexanoic Acid, Malic |
| | Acid, Propionic Acid, Glutamic Acid, Oxalic Acid, Lactic Acid, Propiolic |
| | Acid, Acetic Acid, Valeric Acid, Acetoacetic acid, Pyruvic Acid, Glycolic |
| | Acid, Formic Acid, Succinic Acid, Levuliniuc Acid, Adipitic Acid, 2- |
| | Hydroxyisocaproic Acid, Phthalic Acid, Cinnamic Acid, Acetylsalicylic |
| | Acid, Citric Acid, Isocitric Acid, Fumaric Acid, Gluconic Acid, Gallic |
| | Acid, Benzoic Acid, 4-hydroxybenxoic Acid, Trimesic Acid, Nicotinic |
| | Acid, Cis-Aconitic Acid, Trans-Aconitic Acid, Cinnamic Acid, Vanillic |
| | acid, Salicylic Acid, oxaloacetic acid, mesooxalic acid, alpha-keto-glutaric |
| | acid, beta-keto-glutaric acid, hydroxypyruvic acid, 3-mercaptopyruvic |
| | acid, Aspartic Acid |
| Glutamic Acid | Malonic Acid, Tartaric Acid, Glutaric Acid, Hexanoic Acid, Malic |
| | Acid, Propionic Acid, Glutamic Acid, Oxalic Acid, Salicylic Acid, Lactic |
| | Acid, Propiolic Acid, Acetic Acid, Valeric Acid, Acetoacetic acid, Pyruvic |
| | Acid, Glycolic Acid, Formic Acid, Succinic Acid, Levulinic Acid, Adipitic |
| | Acid, 2-Hydroxyisocaproic Acid, Phthalic Acid, Cinnamic Acid, Acetylsalicylic |
| | Acid, Citric Acid, Isocitric Acid, Fumaric Acid, Gluconic Acid, Gallic |
| | Acid, Benzoic Acid, 4-hydroxybenxoic Acid, Trimesic Acid, Nicotinic |
| | Acid, Cis-Aconitic Acid, Trans-Aconitic Acid, Cinnamic Acid, Vanillic |
| | acid, Aspartic Acid, Glutamic |
| | Acid, Valine, Leucine, Isoleucine, Alanine, Arginine, Lysine, Proline, Cysteine, |
| | Threonine, Methionine, Histidine, Phenylalanine, Tyrosine, Tryptophan, Asparagine, |
| | Glycine, Serine, oxaloacetic acid, mesooxalic acid, alpha-keto-glutaric |
| | acid, beta-keto-glutaric acid, hydroxypyruvic acid, 3-mercaptopyruvic |
| | acid, Aspartic Acid |
| Glutaric Acid | Malonic Acid, Tartaric Acid, Glutaric Acid, Hexanoic Acid, Malic |
| | Acid, Propionic Acid, Glutamic Acid, Oxalic Acid, Salicylic Acid, Lactic |
| | Acid, Propiolic Acid, Acetic Acid, Valeric Acid, Acetoacetic acid, Pyruvic |
| | Acid, Glycolic Acid, Formic Acid, Succinic Acid, Levuliniuc Acid, Adipitic |
| | Acid, 2-Hydroxyisocaproic Acid, Phthalic Acid, Cinnamic Acid, Acetylsalicylic |
| | Acid, Citric Acid, Isocitric Acid, Fumaric Acid, Gluconic Acid, Gallic |
| | Acid, Benzoic Acid, 4-hydroxybenxoic Acid, Trimesic Acid, Nicotinic |
| | Acid, Cis-Aconitic Acid, Trans-Aconitic Acid, Cinnamic Acid, Vanillic |
| | acid, oxaloacetic acid, mesooxalic acid, alpha-keto-glutaric acid, beta-keto- |
| | glutaric acid, hydroxypyruvic acid, 3-mercaptopyruvic acid, Aspartic Acid |
| Glycine | Malonic Acid, Tartaric Acid, Glutaric Acid, Hexanoic Acid, Malic |
| | Acid, Propionic Acid, Glutamic Acid, Oxalic Acid, Salicylic Acid, Lactic |
| | Acid, Propiolic Acid, Acetic Acid, Valeric Acid, Acetoacetic acid, Pyruvic |
| | Acid, Glycolic Acid, Formic Acid, Succinic Acid, Levulinic Acid, Adipitic |
| | Acid, 2-Hydroxyisocaproic Acid, Phthalic Acid, Cinnamic Acid, Acetylsalicylic |
| | Acid, Citric Acid, Isocitric Acid, Fumaric Acid, Gluconic Acid, Gallic |
| | Acid, Benzoic Acid, 4-hydroxybenxoic Acid, Trimesic Acid, Nicotinic |
| | Acid, Cis-Aconitic Acid, Trans-Aconitic Acid, Cinnamic Acid, Vanillic |
| | acid, Aspartic Acid, Glutamic |
| | Acid, Valine, Leucine, Isoleucine, Alanine, Arginine, Lysine, Proline, Cysteine, |
| | Threonine, Methionine, Histidine, Phenylalanine, Tyrosine, Tryptophan, Asparagine, |
| | Glycine, Serine, oxaloacetic acid, mesooxalic acid, alpha-keto-glutaric |
| | acid, beta-keto-glutaric acid, hydroxypyruvic acid, 3-mercaptopyruvic |
| | acid, Aspartic Acid |
| Glycolic Acid | Malonic Acid, Tartaric Acid, Glutaric Acid, Hexanoic Acid, Malic |
| | Acid, Propionic Acid, Glutamic Acid, Oxalic Acid, Lactic Acid, Propiolic |
| | Acid, Acetic Acid, Valeric Acid, Acetoacetic acid, Pyruvic Acid, Glycolic |
| | Acid, Formic Acid, Succinic Acid, Levuliniuc Acid, Adipitic Acid, 2- |
| | Hydroxyisocaproic Acid, Citric Acid, Isocitric Acid, Fumaric |
| | Acid, Acetylsalicylic Acid, Phthalic Acid, Cinnamic Acid, Gluconic Acid, Gallic |
| | Acid, Benzoic Acid, 4-hydroxybenxoic Acid, Trimesic Acid, Nicotinic |
| | Acid, Cis-Aconitic Acid, Trans-Aconitic Acid, Cinnamic Acid, Vanillic |
| | acid, Salicylic Acid, oxaloacetic acid, mesooxalic acid, alpha-keto-glutaric |
| | acid, beta-keto-glutaric acid, hydroxypyruvic acid, 3-mercaptopyruvic |
| | acid, Aspartic Acid |
| Hexanoic Acid | Malonic Acid, Tartaric Acid, Glutaric Acid, Hexanoic Acid, Malic |
| | Acid, Propionic Acid, Glutamic Acid, Oxalic Acid, Salicylic Acid, Lactic |
| | Acid, Propiolic Acid, Acetic Acid, Valeric Acid, Acetoacetic acid, Pyruvic |
| | Acid, Glycolic Acid, Formic Acid, Succinic Acid, Levuliniuc Acid, Adipitic |
| | Acid, 2-Hydroxyisocaproic Acid, Phthalic Acid, Cinnamic Acid, Acetylsalicylic |
| | Acid, Citric Acid, Isocitric Acid, Fumaric Acid, Gluconic Acid, Gallic |
| | Acid, Benzoic Acid, 4-hydroxybenxoic Acid, Trimesic Acid, Nicotinic |
| | Acid, Cis-Aconitic Acid, Trans-Aconitic Acid, Cinnamic Acid, Vanillic |
| | acid, oxaloacetic acid, mesooxalic acid, alpha-keto-glutaric acid, beta-keto- |
| | glutaric acid, hydroxypyruvic acid, 3-mercaptopyruvic acid, Aspartic Acid |
| Histidine | Malonic Acid, Tartaric Acid, Glutaric Acid, Hexanoic Acid, Malic |
| | Acid, Propionic Acid, Glutamic Acid, Oxalic Acid, Salicylic Acid, Lactic |
| | Acid, Propiolic Acid, Acetic Acid, Valeric Acid, Acetoacetic acid, Pyruvic |
| | Acid, Glycolic Acid, Formic Acid, Succinic Acid, Levulinic Acid, Adipitic |
| | Acid, 2-Hydroxyisocaproic Acid, Phthalic Acid, Cinnamic Acid, Acetylsalicylic |
| | Acid, Citric Acid, Isocitric Acid, Fumaric Acid, Gluconic Acid, Gallic |
| | Acid, Benzoic Acid, 4-hydroxybenxoic Acid, Trimesic Acid, Nicotinic |
| | Acid, Cis-Aconitic Acid, Trans-Aconitic Acid, Cinnamic Acid, Vanillic |
| | acid, Aspartic Acid, Glutamic |
| | Acid, Valine, Leucine, Isoleucine, Alanine, Arginine, Lysine, Proline, Cysteine, |
| | Threonine, Methionine, Histidine, Phenylalanine, Tyrosine, Tryptophan, Asparagine, |
| | Glycine, Serine, oxaloacetic acid, mesooxalic acid, alpha-keto-glutaric |
| | acid, beta-keto-glutaric acid, hydroxypyruvic acid, 3-mercaptopyruvic |
| | acid, Aspartic Acid |
| Hydroxypyruvic acid | Malonic Acid, Tartaric Acid, Glutaric Acid, Hexanoic Acid, Malic |
| | Acid, Propionic Acid, Glutamic Acid, Oxalic Acid, Salicylic Acid, Lactic |
| | Acid, Propiolic Acid, Acetic Acid, Valeric Acid, Acetoacetic acid, Pyruvic |
| | Acid, Glycolic Acid, Formic Acid, Succinic Acid, Levulinic Acid, Adipitic |
| | Acid, 2-Hydroxyisocaproic Acid, Phthalic Acid, Cinnamic Acid, Acetylsalicylic |
| | Acid, Salicylic Acid, Citric Acid, Isocitric Acid, Fumaric Acid, Gluconic |
| | Acid, Gallic Acid, Benzoic Acid, 4-hydroxybenxoic Acid, Trimesic |
| | Acid, Nicotinic Acid, Cis-Aconitic Acid, Trans-Aconitic Acid, Cinnamic |
| | Acid, Vanillic acid, oxaloacetic acid, mesooxalic acid, alpha-keto-glutaric |
| | acid, beta-keto-glutaric acid, hydroxypyruvic acid, 3-mercaptopyruvic |
| | acid, Aspartic Acid |
| Isocitric Acid | Malonic Acid, Tartaric Acid, Glutaric Acid, Hexanoic Acid, Malic |
| | Acid, Propionic Acid, Glutamic Acid, Oxalic Acid, Lactic Acid, Propiolic |
| | Acid, Acetic Acid, Valeric Acid, Acetoacetic acid, Pyruvic Acid, Glycolic |
| | Acid, Formic Acid, Succinic Acid, Levuliniuc Acid, Adipitic Acid, 2- |
| | Hydroxyisocaproic Acid, Citric Acid, Isocitric Acid, Fumaric |
| | Acid, Acetylsalicylic Acid, Phthalic Acid, Cinnamic Acid, Gluconic Acid, Gallic |
| | Acid, Benzoic Acid, 4-hydroxybenxoic Acid, Trimesic Acid, Nicotinic |
| | Acid, Cis-Aconitic Acid, Trans-Aconitic Acid, Cinnamic Acid, Vanillic |
| | acid, Salicylic Acid, oxaloacetic acid, mesooxalic acid, alpha-keto-glutaric |
| | acid, beta-keto-glutaric acid, hydroxypyruvic acid, 3-mercaptopyruvic |
| | acid, Aspartic Acid |
| Isoleucine | Malonic Acid, Tartaric Acid, Glutaric Acid, Hexanoic Acid, Malic |
| | Acid, Propionic Acid, Glutamic Acid, Oxalic Acid, Salicylic Acid, Lactic |
| | Acid, Propiolic Acid, Acetic Acid, Valeric Acid, Acetoacetic acid, Pyruvic |
| | Acid, Glycolic Acid, Formic Acid, Succinic Acid, Levulinic Acid, Adipitic |
| | Acid, 2-Hydroxyisocaproic Acid, Phthalic Acid, Cinnamic Acid, Acetylsalicylic |
| | Acid, Citric Acid, Isocitric Acid, Fumaric Acid, Gluconic Acid, Gallic |
| | Acid, Benzoic Acid, 4-hydroxybenxoic Acid, Trimesic Acid, Nicotinic |
| | Acid, Cis-Aconitic Acid, Trans-Aconitic Acid, Cinnamic Acid, Vanillic |
| | acid, Aspartic Acid, Glutamic |
| | Acid, Valine, Leucine, Isoleucine, Alanine, Arginine, Lysine, Proline, Cysteine, |
| | Threonine, Methionine, Histidine, Phenylalanine, Tyrosine, Tryptophan, Asparagine, |
| | Glycine, Serine, mesooxalic acid, alpha-keto-glutaric acid, beta-keto-glutaric |
| | acid, hydroxypyruvic acid, 3-mercaptopyruvic acid, Aspartic Acid |
| alpha- | Malonic Acid, Tartaric Acid, Glutaric Acid, Hexanoic Acid, Malic |
| ketoglutaric acid | Acid, Propionic Acid, Glutamic Acid, Oxalic Acid, Salicylic Acid, Lactic |
| | Acid, Propiolic Acid, Acetic Acid, Valeric Acid, Acetoacetic acid, Pyruvic |
| | Acid, Glycolic Acid, Formic Acid, Succinic Acid, Levulinic Acid, Adipitic |
| | Acid, 2-Hydroxyisocaproic Acid, Phthalic Acid, Cinnamic Acid, Acetylsalicylic |
| | Acid, Citric Acid, Isocitric Acid, Fumaric Acid, Gluconic Acid, Gallic |
| | Acid, Benzoic Acid, 4-hydroxybenxoic Acid, Trimesic Acid, Nicotinic |
| | Acid, Cis-Aconitic Acid, Trans-Aconitic Acid, Cinnamic Acid, Vanillic |
| | acid, oxaloacetic acid, mesooxalic acid, alpha-keto-glutaric acid, beta-keto- |
| | glutaric acid, hydroxypyruvic acid, 3-mercaptopyruvic acid, Aspartic Acid |
| beta- | Malonic Acid, Tartaric Acid, Glutaric Acid, Hexanoic Acid, Malic |
| ketoglutaric acid | Acid, Propionic Acid, Glutamic Acid, Oxalic Acid, Salicylic Acid, Lactic |
| | Acid, Propiolic Acid, Acetic Acid, Valeric Acid, Acetoacetic acid, Pyruvic |
| | Acid, Glycolic Acid, Formic Acid, Succinic Acid, Levulinic Acid, Adipitic |
| | Acid, 2-Hydroxyisocaproic Acid, Phthalic Acid, Cinnamic Acid, Acetylsalicylic |
| | Acid, Citric Acid, Isocitric Acid, Fumaric Acid, Gluconic Acid, Gallic |
| | Acid, Benzoic Acid, 4-hydroxybenxoic Acid, Trimesic Acid, Nicotinic |
| | Acid, Cis-Aconitic Acid, Trans-Aconitic Acid, Cinnamic Acid, Vanillic |
| | acid, oxaloacetic acid, mesooxalic acid, alpha-keto-glutaric acid, beta-keto- |
| | glutaric acid, hydroxypyruvic acid, 3-mercaptopyruvic acid, Aspartic Acid |
| Lactic Acid | Malonic Acid, Tartaric Acid, Glutaric Acid, Hexanoic Acid, Malic |
| | Acid, Propionic Acid, Glutamic Acid, Oxalic Acid, Salicylic Acid, Lactic |
| | Acid, Propiolic Acid, Acetic Acid, Valeric Acid, Acetoacetic acid, Pyruvic |
| | Acid, Glycolic Acid, Formic Acid, Succinic Acid, Levuliniuc Acid, Adipitic |
| | Acid, 2-Hydroxyisocaproic Acid, Phthalic Acid, Cinnamic Acid, Acetylsalicylic |
| | Acid, Citric Acid, Isocitric Acid, Fumaric Acid, Gluconic Acid, Gallic |
| | Acid, Benzoic Acid, 4-hydroxybenxoic Acid, Trimesic Acid, Nicotinic |
| | Acid, Cis-Aconitic Acid, Trans-Aconitic Acid, Cinnamic Acid, Vanillic |
| | acid, oxaloacetic acid, mesooxalic acid, alpha-keto-glutaric acid, beta-keto- |
| | glutaric acid, hydroxypyruvic acid, 3-mercaptopyruvic acid, Aspartic Acid |
| Leucine | Malonic Acid, Tartaric Acid, Glutaric Acid, Hexanoic Acid, Malic |
| | Acid, Propionic Acid, Glutamic Acid, Oxalic Acid, Salicylic Acid, Lactic |
| | Acid, Propiolic Acid, Acetic Acid, Valeric Acid, Acetoacetic acid, Pyruvic |
| | Acid, Glycolic Acid, Formic Acid, Succinic Acid, Levulinic Acid, Adipitic |
| | Acid, 2-Hydroxyisocaproic Acid, Phthalic Acid, Cinnamic Acid, Acetylsalicylic |
| | Acid, Citric Acid, Isocitric Acid, Fumaric Acid, Gluconic Acid, Gallic |
| | Acid, Benzoic Acid, 4-hydroxybenxoic Acid, Trimesic Acid, Nicotinic |
| | Acid, Cis-Aconitic Acid, Trans-Aconitic Acid, Cinnamic Acid, Vanillic |
| | acid, Aspartic Acid, Glutamic |
| | Acid, Valine, Leucine, Isoleucine, Alanine, Arginine, Lysine, Proline, Cysteine, |
| | Threonine, Methionine, Histidine, Phenylalanine, Tyrosine, Tryptophan, Asparagine, |
| | Glycine, Serine, oxaloacetic acid, mesooxalic acid, alpha-keto-glutaric |
| | acid, beta-keto-glutaric acid, hydroxypyruvic acid, 3-mercaptopyruvic |
| | acid, Aspartic Acid |
| Levuliniuc Acid | Malonic Acid, Tartaric Acid, Glutaric Acid, Hexanoic Acid, Malic |
| | Acid, Propionic Acid, Glutamic Acid, Oxalic Acid, Salicylic Acid, Lactic |
| | Acid, Propiolic Acid, Acetic Acid, Valeric Acid, Acetoacetic acid, Pyruvic |
| | Acid, Glycolic Acid, Formic Acid, Succinic Acid, Levuliniuc Acid, Adipitic |
| | Acid, 2-Hydroxyisocaproic Acid, Phthalic Acid, Cinnamic Acid, Acetylsalicylic |
| | Acid, Citric Acid, Isocitric Acid, Fumaric Acid, Gluconic Acid, Gallic |
| | Acid, Benzoic Acid, 4-hydroxybenxoic Acid, Trimesic Acid, Nicotinic |
| | Acid, Cis-Aconitic Acid, Trans-Aconitic Acid, Cinnamic Acid, Vanillic |
| | acid, oxaloacetic acid, mesooxalic acid, alpha-keto-glutaric acid, beta-keto- |
| | glutaric acid, hydroxypyruvic acid, 3-mercaptopyruvic acid, Aspartic Acid |
| Lysine | Malonic Acid, Tartaric Acid, Glutaric Acid, Hexanoic Acid, Malic |
| | Acid, Propionic Acid, Glutamic Acid, Oxalic Acid, Salicylic Acid, Lactic |
| | Acid, Propiolic Acid, Acetic Acid, Valeric Acid, Acetoacetic acid, Pyruvic |
| | Acid, Glycolic Acid, Formic Acid, Succinic Acid, Levulinic Acid, Adipitic |
| | Acid, 2-Hydroxyisocaproic Acid, Phthalic Acid, Cinnamic Acid, Acetylsalicylic |
| | Acid, Citric Acid, Isocitric Acid, Fumaric Acid, Gluconic Acid, Gallic |
| | Acid, Benzoic Acid, 4-hydroxybenxoic Acid, Trimesic Acid, Nicotinic |
| | Acid, Cis-Aconitic Acid, Trans-Aconitic Acid, Cinnamic Acid, Vanillic |
| | acid, Aspartic Acid, Glutamic |
| | Acid, Valine, Leucine, Isoleucine, Alanine, Arginine, Lysine, Proline, Cysteine, |
| | Threonine, Methionine, Histidine, Phenylalanine, Tyrosine, Tryptophan, Asparagine, |
| | Glycine, Serine, oxaloacetic acid, mesooxalic acid, alpha-keto-glutaric |
| | acid, beta-keto-glutaric acid, hydroxypyruvic acid, 3-mercaptopyruvic |
| | acid, Aspartic Acid |
| Malic Acid | Malonic Acid, Tartaric Acid, Glutaric Acid, Hexanoic Acid, Malic |
| | Acid, Propionic Acid, Glutamic Acid, Oxalic Acid, Salicylic Acid, Lactic |
| | Acid, Propiolic Acid, Acetic Acid, Valeric Acid, Acetoacetic acid, Pyruvic |
| | Acid, Glycolic Acid, Formic Acid, Succinic Acid, Levuliniuc Acid, Adipitic |
| | Acid, 2-Hydroxyisocaproic Acid, Phthalic Acid, Cinnamic Acid, Acetylsalicylic |
| | Acid, Citric Acid, Isocitric Acid, Fumaric Acid, Gluconic Acid, Gallic |
| | Acid, Benzoic Acid, 4-hydroxybenxoic Acid, Trimesic Acid, Nicotinic |
| | Acid, Cis-Aconitic Acid, Trans-Aconitic Acid, Cinnamic Acid, Vanillic |
| | acid, oxaloacetic acid, mesooxalic acid, alpha-keto-glutaric acid, beta-keto- |
| | glutaric acid, hydroxypyruvic acid, 3-mercaptopyruvic acid, Aspartic Acid |
| Malonic Acid | Malonic Acid, Tartaric Acid, Glutaric Acid, Hexanoic Acid, Malic |
| | Acid, Propionic Acid, Glutamic Acid, Oxalic Acid, Salicylic Acid, Lactic |
| | Acid, Propiolic Acid, Acetic Acid, Valeric Acid, Acetoacetic acid, Pyruvic |
| | Acid, Glycolic Acid, Formic Acid, Succinic Acid, Levuliniuc Acid, Adipitic |
| | Acid, 2-Hydroxyisocaproic Acid, Phthalic Acid, Cinnamic Acid, Acetylsalicylic |
| | Acid, Citric Acid, Isocitric Acid, Fumaric Acid, Gluconic Acid, Gallic |
| | Acid, Benzoic Acid, 4-hydroxybenxoic Acid, Trimesic Acid, Nicotinic |
| | Acid, Cis-Aconitic Acid, Trans-Aconitic Acid, Cinnamic Acid, Vanillic |
| | acid, oxaloacetic acid, mesooxalic acid, alpha-keto-glutaric acid, beta-keto- |
| | glutaric acid, hydroxypyruvic acid, 3-mercaptopyruvic acid, Aspartic Acid |
| Mesooxalic acid | Malonic Acid, Tartaric Acid, Glutaric Acid, Hexanoic Acid, Malic |
| | Acid, Propionic Acid, Glutamic Acid, Oxalic Acid, Salicylic Acid, Lactic |
| | Acid, Propiolic Acid, Acetic Acid, Valeric Acid, Acetoacetic acid, Pyruvic |
| | Acid, Glycolic Acid, Formic Acid, Succinic Acid, Levulinic Acid, Adipitic |
| | Acid, 2-Hydroxyisocaproic Acid, Phthalic Acid, Cinnamic Acid, Acetylsalicylic |
| | Acid, Salicylic Acid, Citric Acid, Isocitric Acid, Fumaric Acid, Gluconic |
| | Acid, Gallic Acid, Benzoic Acid, 4-hydroxybenxoic Acid, Trimesic |
| | Acid, Nicotinic Acid, Cis-Aconitic Acid, Trans-Aconitic Acid, Cinnamic |
| | Acid, Vanillic acid, oxaloacetic acid, mesooxalic acid, alpha-keto-glutaric |
| | acid, beta-keto-glutaric acid, hydroxypyruvic acid, 3-mercaptopyruvic |
| | acid, Aspartic Acid |
| Methionine | Malonic Acid, Tartaric Acid, Glutaric Acid, Hexanoic Acid, Malic |
| | Acid, Propionic Acid, Glutamic Acid, Oxalic Acid, Salicylic Acid, Lactic |
| | Acid, Propiolic Acid, Acetic Acid, Valeric Acid, Acetoacetic acid, Pyruvic |
| | Acid, Glycolic Acid, Formic Acid, Succinic Acid, Levulinic Acid, Adipitic |
| | Acid, 2-Hydroxyisocaproic Acid, Phthalic Acid, Cinnamic Acid, Acetylsalicylic |
| | Acid, Citric Acid, Isocitric Acid, Fumaric Acid, Gluconic Acid, Gallic |
| | Acid, Benzoic Acid, 4-hydroxybenxoic Acid, Trimesic Acid, Nicotinic |
| | Acid, Cis-Aconitic Acid, Trans-Aconitic Acid, Cinnamic Acid, Vanillic |
| | acid, Aspartic Acid, Glutamic |
| | Acid, Valine, Leucine, Isoleucine, Alanine, Arginine, Lysine, Proline, Cysteine, |
| | Threonine, Methionine, Histidine, Phenylalanine, Tyrosine, Tryptophan, Asparagine, |
| | Glycine, Serine, oxaloacetic acid, mesooxalic acid, alpha-keto-glutaric |
| | acid, beta-keto-glutaric acid, hydroxypyruvic acid, 3-mercaptopyruvic |
| | acid, Aspartic Acid |
| Nicotinic Acid | Malonic Acid, Tartaric Acid, Glutaric Acid, Hexanoic Acid, Malic |
| | Acid, Propionic Acid, Glutamic Acid, Oxalic Acid, Lactic Acid, Propiolic |
| | Acid, Acetic Acid, Nicotinic Acid, Valeric Acid, Acetoacetic acid, Pyruvic |
| | Acid, Glycolic Acid, Formic Acid, Succinic Acid, Levuliniuc Acid, Adipitic |
| | Acid, 2-Hydroxyisocaproic Acid, Citric Acid, Isocitric Acid, Fumaric |
| | Acid, oxaloacetic acid, mesooxalic acid, alpha-keto-glutaric acid, beta-keto- |
| | glutaric acid, hydroxypyruvic acid, 3-mercaptopyruvic acid, Aspartic Acid |
| Oxalic Acid | Malonic Acid, Tartaric Acid, Glutaric Acid, Hexanoic Acid, Malic |
| | Acid, Propionic Acid, Glutamic Acid, Oxalic Acid, Salicylic Acid, Lactic |
| | Acid, Propiolic Acid, Acetic Acid, Valeric Acid, Acetoacetic acid, Pyruvic |
| | Acid, Glycolic Acid, Formic Acid, Succinic Acid, Levuliniuc Acid, Adipitic |
| | Acid, 2-Hydroxyisocaproic Acid, Phthalic Acid, Cinnamic Acid, Acetylsalicylic |
| | Acid, Citric Acid, Isocitric Acid, Fumaric Acid, Gluconic Acid, Gallic |
| | Acid, Benzoic Acid, 4-hydroxybenxoic Acid, Trimesic Acid, Nicotinic |
| | Acid, Cis-Aconitic Acid, Trans-Aconitic Acid, Cinnamic Acid, Vanillic |
| | acid, oxaloacetic acid, mesooxalic acid, alpha-keto-glutaric acid, beta-keto- |
| | glutaric acid, hydroxypyruvic acid, 3-mercaptopyruvic acid, Aspartic Acid |
| Oxaloacetic acid | Malonic Acid, Tartaric Acid, Glutaric Acid, Hexanoic Acid, Malic |
| | Acid, Propionic Acid, Glutamic Acid, Oxalic Acid, Salicylic Acid, Lactic |
| | Acid, Propiolic Acid, Acetic Acid, Valeric Acid, Acetoacetic acid, Pyruvic |
| | Acid, Glycolic Acid, Formic Acid, Succinic Acid, Levuliniuc Acid, Adipitic |
| | Acid, 2-Hydroxyisocaproic Acid, Phthalic Acid, Cinnamic Acid, Acetylsalicylic |
| | Acid, Citric Acid, Isocitric Acid, Fumaric Acid, Gluconic Acid, Gallic |
| | Acid, Benzoic Acid, 4-hydroxybenxoic Acid, Trimesic Acid, Nicotinic |
| | Acid, Cis-Aconitic Acid, Trans-Aconitic Acid, Cinnamic Acid, Vanillic |
| | acid, oxaloacetic acid, mesooxalic acid, alpha-keto-glutaric acid, beta-keto- |
| | glutaric acid, hydroxypyruvic acid, 3-mercaptopyruvic acid, Aspartic Acid |
| Phenylalanine | Malonic Acid, Tartaric Acid, Glutaric Acid, Hexanoic Acid, Malic |
| | Acid, Propionic Acid, Glutamic Acid, Oxalic Acid, Salicylic Acid, Lactic |
| | Acid, Propiolic Acid, Acetic Acid, Valeric Acid, Acetoacetic acid, Pyruvic |
| | Acid, Glycolic Acid, Formic Acid, Succinic Acid, Levulinic Acid, Adipitic |
| | Acid, 2-Hydroxyisocaproic Acid, Phthalic Acid, Cinnamic Acid, Acetylsalicylic |
| | Acid, Citric Acid, Isocitric Acid, Fumaric Acid, Gluconic Acid, Gallic |
| | Acid, Benzoic Acid, 4-hydroxybenxoic Acid, Trimesic Acid, Nicotinic |
| | Acid, Cis-Aconitic Acid, Trans-Aconitic Acid, Cinnamic Acid, Vanillic |
| | acid, Aspartic Acid, Glutamic |
| | Acid, Valine, Leucine, Isoleucine, Alanine, Arginine, Lysine, Proline, Cysteine, |
| | Threonine, Methionine, Histidine, Phenylalanine, Tyrosine, Tryptophan, Asparagine, |
| | Glycine, Serine, oxaloacetic acid, mesooxalic acid, alpha-keto-glutaric |
| | acid, beta-keto-glutaric acid, hydroxypyruvic acid, 3-mercaptopyruvic |
| | acid, Aspartic Acid |
| Phthalic Acid | Malonic Acid, Tartaric Acid, Glutaric Acid, Hexanoic Acid, Malic |
| | Acid, Propionic Acid, Glutamic Acid, Oxalic Acid, Lactic Acid, Propiolic |
| | Acid, Acetic Acid, Valeric Acid, Acetoacetic acid, Pyruvic Acid, Glycolic |
| | Acid, Formic Acid, Succinic Acid, Levuliniuc Acid, Adipitic Acid, 2- |
| | Hydroxyisocaproic Acid, Citric Acid, Isocitric Acid, Fumaric Acid, oxaloacetic |
| | acid, mesooxalic acid, alpha-keto-glutaric acid, beta-keto-glutaric |
| | acid, hydroxypyruvic acid, 3-mercaptopyruvic acid, Aspartic Acid |
| Proline | Malonic Acid, Tartaric Acid, Glutaric Acid, Hexanoic Acid, Malic |
| | Acid, Propionic Acid, Glutamic Acid, Oxalic Acid, Salicylic Acid, Lactic |
| | Acid, Propiolic Acid, Acetic Acid, Valeric Acid, Acetoacetic acid, Pyruvic |
| | Acid, Glycolic Acid, Formic Acid, Succinic Acid, Levulinic Acid, Adipitic |
| | Acid, 2-Hydroxyisocaproic Acid, Phthalic Acid, Cinnamic Acid, Acetylsalicylic |
| | Acid, Citric Acid, Isocitric Acid, Fumaric Acid, Gluconic Acid, Gallic |
| | Acid, Benzoic Acid, 4-hydroxybenxoic Acid, Trimesic Acid, Nicotinic |
| | Acid, Cis-Aconitic Acid, Trans-Aconitic Acid, Cinnamic Acid, Vanillic |
| | acid, Aspartic Acid, Glutamic |
| | Acid, Valine, Leucine, Isoleucine, Alanine, Arginine, Lysine, Proline, Cysteine, |
| | Threonine, Methionine, Histidine, Phenylalanine, Tyrosine, Tryptophan, Asparagine, |
| | Glycine, oxaloacetic acid, mesooxalic acid, alpha-keto-glutaric acid, beta-keto- |
| | glutaric acid, hydroxypyruvic acid, 3-mercaptopyruvic acid, Aspartic Acid |
| Propiolic Acid | Malonic Acid, Tartaric Acid, Glutaric Acid, Hexanoic Acid, Malic |
| | Acid, Propionic Acid, Glutamic Acid, Oxalic Acid, Lactic Acid, Propiolic |
| | Acid, Acetic Acid, Valeric Acid, Acetoacetic acid, Pyruvic Acid, Glycolic |
| | Acid, Formic Acid, Succinic Acid, Levuliniuc Acid, Adipitic Acid, 2- |
| | Hydroxyisocaproic Acid, Phthalic Acid, Cinnamic Acid, Acetylsalicylic |
| | Acid, Citric Acid, Isocitric Acid, Fumaric Acid, Gluconic Acid, Gallic |
| | Acid, Benzoic Acid, 4-hydroxybenxoic Acid, Trimesic Acid, Nicotinic |
| | Acid, Cis-Aconitic Acid, Trans-Aconitic Acid, Cinnamic Acid, Vanillic |
| | acid, Salicylic Acid, oxaloacetic acid, mesooxalic acid, alpha-keto-glutaric |
| | acid, beta-keto-glutaric acid, hydroxypyruvic acid, 3-mercaptopyruvic |
| | acid, Aspartic Acid |
| Propionic Acid | Malonic Acid, Tartaric Acid, Glutaric Acid, Hexanoic Acid, Malic |
| | Acid, Propionic Acid, Glutamic Acid, Oxalic Acid, Salicylic Acid, Lactic |
| | Acid, Propiolic Acid, Acetic Acid, Valeric Acid, Acetoacetic acid, Pyruvic |
| | Acid, Glycolic Acid, Formic Acid, Succinic Acid, Levuliniuc Acid, Adipitic |
| | Acid, 2-Hydroxyisocaproic Acid, Phthalic Acid, Cinnamic Acid, Acetylsalicylic |
| | Acid, Citric Acid, Isocitric Acid, Fumaric Acid, Gluconic Acid, Gallic |
| | Acid, Benzoic Acid, 4-hydroxybenxoic Acid, Trimesic Acid, Nicotinic |
| | Acid, Cis-Aconitic Acid, Trans-Aconitic Acid, Cinnamic Acid, Vanillic |
| | acid, oxaloacetic acid, mesooxalic acid, alpha-keto-glutaric acid, beta-keto- |
| | glutaric acid, hydroxypyruvic acid, 3-mercaptopyruvic acid, Aspartic Acid |
| Pyruvic Acid | Malonic Acid, Tartaric Acid, Glutaric Acid, Hexanoic Acid, Malic |
| | Acid, Propionic Acid, Glutamic Acid, Oxalic Acid, Salicylic Acid, Lactic |
| | Acid, Propiolic Acid, Acetic Acid, Valeric Acid, Acetoacetic acid, Pyruvic |
| | Acid, Glycolic Acid, Formic Acid, Succinic Acid, Levuliniuc Acid, Adipitic |
| | Acid, 2-Hydroxyisocaproic Acid, Phthalic Acid, Cinnamic Acid, Acetylsalicylic |
| | Acid, Citric Acid, Isocitric Acid, Fumaric Acid, Gluconic Acid, Gallic |
| | Acid, Benzoic Acid, 4-hydroxybenxoic Acid, Trimesic Acid, Nicotinic |
| | Acid, Cis-Aconitic Acid, Trans-Aconitic Acid, Cinnamic Acid, Vanillic |
| | acid, oxaloacetic acid, mesooxalic acid, alpha-keto-glutaric acid, beta-keto- |
| | glutaric acid, hydroxypyruvic acid, 3-mercaptopyruvic acid, Aspartic Acid |
| Salicylic Acid | Malonic Acid, Tartaric Acid, Glutaric Acid, Hexanoic Acid, Malic |
| | Acid, Propionic Acid, Glutamic Acid, Oxalic Acid, Salicylic Acid, Lactic |
| | Acid, Propiolic Acid, Acetic Acid, Valeric Acid, Acetoacetic acid, Pyruvic |
| | Acid, Glycolic Acid, Formic Acid, Succinic Acid, Levuliniuc Acid, Adipitic |
| | Acid, 2-Hydroxyisocaproic Acid, Phthalic Acid, Cinnamic Acid, Acetylsalicylic |
| | Acid, Citric Acid, Isocitric Acid, Fumaric Acid, Gluconic Acid, Gallic |
| | Acid, Benzoic Acid, 4-hydroxybenxoic Acid, Trimesic Acid, Nicotinic |
| | Acid, Cis-Aconitic Acid, Trans-Aconitic Acid, Cinnamic Acid, Vanillic |
| | acid, oxaloacetic acid, mesooxalic acid, alpha-keto-glutaric acid, beta-keto- |
| | glutaric acid, hydroxypyruvic acid, 3-mercaptopyruvic acid, Aspartic Acid |
| Serine | Malonic Acid, Tartaric Acid, Glutaric Acid, Hexanoic Acid, Malic |
| | Acid, Propionic Acid, Glutamic Acid, Oxalic Acid, Salicylic Acid, Lactic |
| | Acid, Propiolic Acid, Acetic Acid, Valeric Acid, Acetoacetic acid, Pyruvic |
| | Acid, Glycolic Acid, Formic Acid, Succinic Acid, Levulinic Acid, Adipitic |
| | Acid, 2-Hydroxyisocaproic Acid, Phthalic Acid, Cinnamic Acid, Acetylsalicylic |
| | Acid, Citric Acid, Isocitric Acid, Fumaric Acid, Gluconic Acid, Gallic |
| | Acid, Benzoic Acid, 4-hydroxybenxoic Acid, Trimesic Acid, Nicotinic |
| | Acid, Cis-Aconitic Acid, Trans-Aconitic Acid, Cinnamic Acid, Vanillic |
| | acid, Aspartic Acid, Glutamic |
| | Acid, Valine, Leucine, Isoleucine, Alanine, Arginine, Lysine, Proline, Cysteine, |
| | Threonine, Methionine, Histidine, Phenylalanine, Tyrosine, Tryptophan, Asparagine, |
| | Glycine, Serine, oxaloacetic acid, mesooxalic acid, alpha-keto-glutaric |
| | acid, beta-keto-glutaric acid, hydroxypyruvic acid, 3-mercaptopyruvic |
| | acid, Aspartic Acid |
| Succinic Acid | Malonic Acid, Tartaric Acid, Glutaric Acid, Hexanoic Acid, Malic |
| | Acid, Propionic Acid, Glutamic Acid, Oxalic Acid, Lactic Acid, Propiolic |
| | Acid, Acetic Acid, Valeric Acid, Acetoacetic acid, Pyruvic Acid, Glycolic |
| | Acid, Formic Acid, Succinic Acid, Levuliniuc Acid, Adipitic Acid, 2- |
| | Hydroxyisocaproic Acid, Phthalic Acid, Cinnamic Acid, Acetylsalicylic |
| | Acid, Citric Acid, Isocitric Acid, Fumaric Acid, Gluconic Acid, Gallic |
| | Acid, Benzoic Acid, 4-hydroxybenxoic Acid, Trimesic Acid, Nicotinic |
| | Acid, Cis-Aconitic Acid, Trans-Aconitic Acid, Cinnamic Acid, Vanillic |
| | acid, Salicylic Acid, oxaloacetic acid, mesooxalic acid, alpha-keto-glutaric |
| | acid, beta-keto-glutaric acid, hydroxypyruvic acid, 3-mercaptopyruvic |
| | acid, Aspartic Acid |
| Tartaric Acid | Malonic Acid, Tartaric Acid, Glutaric Acid, Hexanoic Acid, Malic |
| | Acid, Propionic Acid, Glutamic Acid, Oxalic Acid, Salicylic Acid, Lactic |
| | Acid, Propiolic Acid, Acetic Acid, Valeric Acid, Acetoacetic acid, Pyruvic |
| | Acid, Glycolic Acid, Formic Acid, Succinic Acid, Levuliniuc Acid, Adipitic |
| | Acid, 2-Hydroxyisocaproic Acid, Phthalic Acid, Cinnamic Acid, Acetylsalicylic |
| | Acid, Citric Acid, Isocitric Acid, Fumaric Acid, Gluconic Acid, Gallic |
| | Acid, Benzoic Acid, 4-hydroxybenxoic Acid, Trimesic Acid, Nicotinic |
| | Acid, Cis-Aconitic Acid, Trans-Aconitic Acid, Cinnamic Acid, Vanillic |
| | acid, oxaloacetic acid, mesooxalic acid, alpha-keto-glutaric acid, beta-keto- |
| | glutaric acid, hydroxypyruvic acid, 3-mercaptopyruvic acid, Aspartic Acid |
| Threonine | Malonic Acid, Tartaric Acid, Glutaric Acid, Hexanoic Acid, Malic |
| | Acid, Propionic Acid, Glutamic Acid, Oxalic Acid, Salicylic Acid, Lactic |
| | Acid, Propiolic Acid, Acetic Acid, Valeric Acid, Acetoacetic acid, Pyruvic |
| | Acid, Glycolic Acid, Formic Acid, Succinic Acid, Levulinic Acid, Adipitic |
| | Acid, 2-Hydroxyisocaproic Acid, Phthalic Acid, Cinnamic Acid, Acetylsalicylic |
| | Acid, Citric Acid, Isocitric Acid, Fumaric Acid, Gluconic Acid, Gallic |
| | Acid, Benzoic Acid, 4-hydroxybenxoic Acid, Trimesic Acid, Nicotinic |
| | Acid, Cis-Aconitic Acid, Trans-Aconitic Acid, Cinnamic Acid, Vanillic |
| | acid, Aspartic Acid, Glutamic |
| | Acid, Valine, Leucine, Isoleucine, Alanine, Arginine, Lysine, Proline, Cysteine, |
| | Threonine, Methionine, Histidine, Phenylalanine, Tyrosine, Tryptophan, Asparagine, |
| | Glycine, Serine, mesooxalic acid, alpha-keto-glutaric acid, beta-keto-glutaric |
| | acid, hydroxypyruvic acid, 3-mercaptopyruvic acid, Aspartic Acid |
| Trans-Aconitic Acid | Malonic Acid, Tartaric Acid, Glutaric Acid, Hexanoic Acid, Malic |
| | Acid, Propionic Acid, Glutamic Acid, Oxalic Acid, Lactic Acid, Propiolic |
| | Acid, Acetic Acid, Valeric Acid, Acetoacetic acid, Pyruvic Acid, Glycolic |
| | Acid, Formic Acid, Succinic Acid, Levuliniuc Acid, Adipitic Acid, 2- |
| | Hydroxyisocaproic Acid, Citric Acid, Isocitric Acid, Fumaric Acid, oxaloacetic |
| | acid, mesooxalic acid, alpha-keto-glutaric acid, beta-keto-glutaric |
| | acid, hydroxypyruvic acid, 3-mercaptopyruvic acid, Trans-Aconitic |
| | Acid, Aspartic Acid |
| Trimesic Acid | Malonic Acid, Tartaric Acid, Glutaric Acid, Hexanoic Acid, Malic |
| | Acid, Propionic Acid, Glutamic Acid, Oxalic Acid, Lactic Acid, Propiolic |
| | Acid, Acetic Acid, Valeric Acid, Acetoacetic acid, Pyruvic Acid, Glycolic |
| | Acid, Formic Acid, Succinic Acid, Levuliniuc Acid, Adipitic Acid, 2- |
| | Hydroxyisocaproic Acid, Citric Acid, Isocitric Acid, Fumaric Acid, Trimesic |
| | acid, oxaloacetic acid, mesooxalic acid, alpha-keto-glutaric acid, beta-keto- |
| | glutaric acid, hydroxypyruvic acid, 3-mercaptopyruvic acid, Aspartic Acid |
| Tryptophan | Malonic Acid, Tartaric Acid, Glutaric Acid, Hexanoic Acid, Malic |
| | Acid, Propionic Acid, Glutamic Acid, Oxalic Acid, Salicylic Acid, Lactic |
| | Acid, Propiolic Acid, Acetic Acid, Valeric Acid, Acetoacetic acid, Pyruvic |
| | Acid, Glycolic Acid, Formic Acid, Succinic Acid, Levulinic Acid, Adipitic |
| | Acid, 2-Hydroxyisocaproic Acid, Phthalic Acid, Cinnamic Acid, Acetylsalicylic |
| | Acid, Citric Acid, Isocitric Acid, Fumaric Acid, Gluconic Acid, Gallic |
| | Acid, Benzoic Acid, 4-hydroxybenxoic Acid, Trimesic Acid, Nicotinic |
| | Acid, Cis-Aconitic Acid, Trans-Aconitic Acid, Cinnamic Acid, Vanillic |
| | acid, Aspartic Acid, Glutamic |
| | Acid, Valine, Leucine, Isoleucine, Alanine, Arginine, Lysine, Proline, Cysteine, |
| | Threonine, Methionine, Histidine, Phenylalanine, Tyrosine, Tryptophan, Asparagine, |
| | Glycine, Serine, oxaloacetic acid, mesooxalic acid, alpha-keto-glutaric |
| | acid, beta-keto-glutaric acid, hydroxypyruvic acid, 3-mercaptopyruvic |
| | acid, Aspartic Acid |
| Tyrosine | Malonic Acid, Tartaric Acid, Glutaric Acid, Hexanoic Acid, Malic |
| | Acid, Propionic Acid, Glutamic Acid, Oxalic Acid, Salicylic Acid, Lactic |
| | Acid, Propiolic Acid, Acetic Acid, Valeric Acid, Acetoacetic acid, Pyruvic |
| | Acid, Glycolic Acid, Formic Acid, Succinic Acid, Levulinic Acid, Adipitic |
| | Acid, 2-Hydroxyisocaproic Acid, Phthalic Acid, Cinnamic Acid, Acetylsalicylic |
| | Acid, Citric Acid, Isocitric Acid, Fumaric Acid, Gluconic Acid, Gallic |
| | Acid, Benzoic Acid, 4-hydroxybenxoic Acid, Trimesic Acid, Nicotinic |
| | Acid, Cis-Aconitic Acid, Trans-Aconitic Acid, Cinnamic Acid, Vanillic |
| | acid, Aspartic Acid, Glutamic |
| | Acid, Valine, Leucine, Isoleucine, Alanine, Arginine, Lysine, Proline, Cysteine, |
| | Threonine, Methionine, Histidine, Phenylalanine, Tyrosine, Tryptophan, Asparagine, |
| | Glycine, Serine, oxaloacetic acid, mesooxalic acid, alpha-keto-glutaric |
| | acid, beta-keto-glutaric acid, hydroxypyruvic acid, 3-mercaptopyruvic |
| | acid, Aspartic Acid |
| Valeric Acid | Malonic Acid, Tartaric Acid, Glutaric Acid, Hexanoic Acid, Malic |
| | Acid, Propionic Acid, Glutamic Acid, Oxalic Acid, Lactic Acid, Propiolic |
| | Acid, Acetic Acid, Valeric Acid, Acetoacetic acid, Pyruvic Acid, Glycolic |
| | Acid, Formic Acid, Succinic Acid, Levuliniuc Acid, Adipitic Acid, 2- |
| | Hydroxyisocaproic Acid, Citric Acid, Isocitric Acid, Fumaric |
| | Acid, Acetylsalicylic Acid, Phthalic Acid, Cinnamic Acid, Gluconic Acid, Gallic |
| | Acid, Benzoic Acid, 4-hydroxybenxoic Acid, Trimesic Acid, Nicotinic |
| | Acid, Cis-Aconitic Acid, Trans-Aconitic Acid, Cinnamic Acid, Vanillic |
| | acid, Salicylic Acid, oxaloacetic acid, mesooxalic acid, alpha-keto-glutaric |
| | acid, beta-keto-glutaric acid, hydroxypyruvic acid, 3-mercaptopyruvic |
| | acid, Aspartic Acid |
| Valine | Malonic Acid, Tartaric Acid, Glutaric Acid, Hexanoic Acid, Malic |
| | Acid, Propionic Acid, Glutamic Acid, Oxalic Acid, Salicylic Acid, Lactic |
| | Acid, Propiolic Acid, Acetic Acid, Valeric Acid, Acetoacetic acid, Pyruvic |
| | Acid, Glycolic Acid, Formic Acid, Succinic Acid, Levulinic Acid, Adipitic |
| | Acid, 2-Hydroxyisocaproic Acid, Phthalic Acid, Cinnamic Acid, Acetylsalicylic |
| | Acid, Citric Acid, Isocitric Acid, Fumaric Acid, Gluconic Acid, Gallic |
| | Acid, Benzoic Acid, 4-hydroxybenxoic Acid, Trimesic Acid, Nicotinic |
| | Acid, Cis-Aconitic Acid, Trans-Aconitic Acid, Cinnamic Acid, Vanillic |
| | acid, Aspartic Acid, Glutamic |
| | Acid, Valine, Leucine, Isoleucine, Alanine, Arginine, Lysine, Proline, Cysteine, |
| | Threonine, Methionine, Histidine, Phenylalanine, Tyrosine, Tryptophan, Asparagine, |
| | Glycine, Serine, oxaloacetic acid, mesooxalic acid, alpha-keto-glutaric |
| | acid, beta-keto-glutaric acid, hydroxypyruvic acid, 3-mercaptopyruvic |
| | acid, Aspartic Acid |
| Vanillic acid | Malonic Acid, Tartaric Acid, Glutaric Acid, Hexanoic Acid, Malic |
| | Acid, Propionic Acid, Glutamic Acid, Oxalic Acid, Lactic Acid, Propiolic |
| | Acid, Acetic Acid, Valeric Acid, Acetoacetic acid, Pyruvic Acid, Glycolic |
| | Acid, Formic Acid, Succinic Acid, Levuliniuc Acid, Adipitic Acid, 2- |
| | Hydroxyisocaproic Acid, Citric Acid, Isocitric Acid, Fumaric |
| | Acid, Acetylsalicylic Acid, Phthalic Acid, Cinnamic Acid, Gluconic Acid, Gallic |
| | Acid, Benzoic Acid, 4-hydroxybenxoic Acid, Trimesic Acid, Nicotinic |
| | Acid, Cis-Aconitic Acid, Trans-Aconitic Acid, Cinnamic Acid, Vanillic |
| | acid, Salicylic Acid, oxaloacetic acid, mesooxalic acid, alpha-keto-glutaric |
| | acid, beta-keto-glutaric acid, hydroxypyruvic acid, 3-mercaptopyruvic |
| | acid, Aspartic Acid |

In certain embodiments, an aromatic or branched may have an effect of sterically hindering a bonding of a second organic acid to the pyridine nitrogen center of nicotine. Examples of suitable organic acid pairs are shown in Table 1.

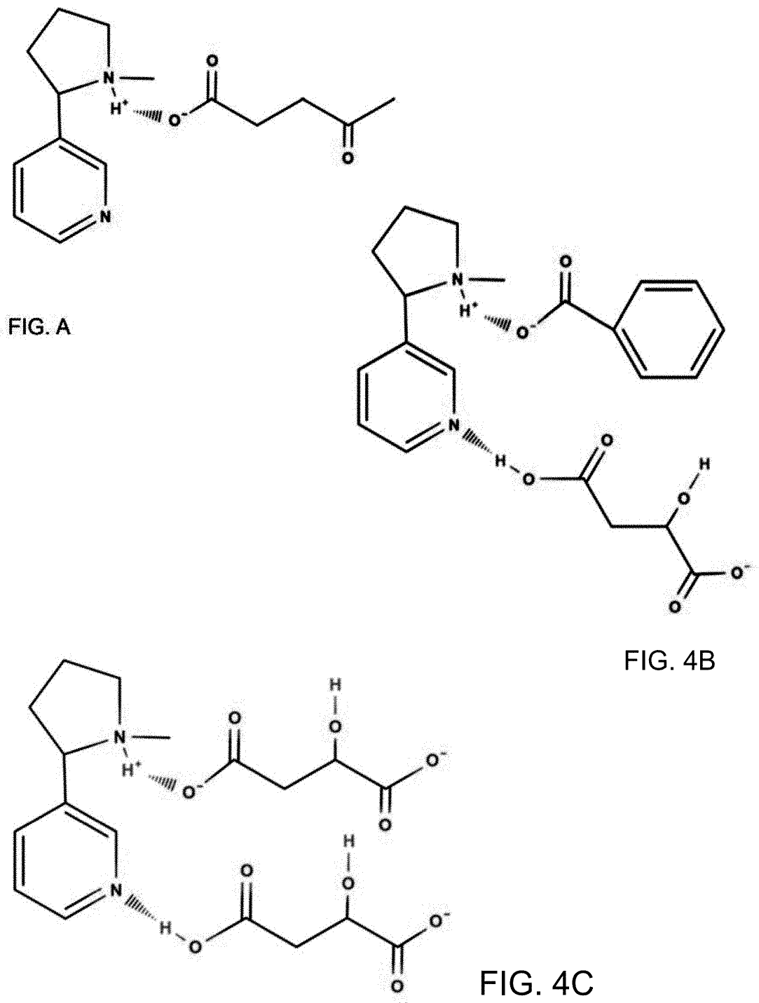

In certain embodiments, heterogeneous nicotine salt complexes are formed utilizing specific methods to select binding “pairs.” For example, in certain embodiments, if the organic acid molecule(s) do not possess competing functional groups that would repel each other into an unfavorable conformation, the higher order salt will not form. In certain embodiments, organic acids are selected containing functional groups that do repel each other (e.g., (+/−) functional group pairs for opposite organic acids. In certain embodiments, if both binding pair are sterically compatible by way of their functional groups, total number and arrangement of carbons, and electrical environment (sigma vs pi bonds leading to distribution of electron density), the higher order salt are formed. An embodied heterogenous nicotine salt complex includes: nicotine N-malate-N′-benzoate (FIG. 4B).

An example of unlikely to form higher order salt, blocked by functional group steric hindrance is nicotine N-trimesate-N′-citrate.

In certain embodiments, compounds that possess free electron density within their functional groups aside from the carboxylate bound to nicotine by ionic forces or hydrogen bonding, will yield a salt that is stronger in “throat hit.” In certain embodiments, the experience is characterized as “bite.” In certain embodiments, an experience is provided ranging from “bite” to “smooth” and points, values, markers, indicia, etc. in a range therebetween. Certain embodiments, provide for nicotine salt containing solution(s) supplied for delivery modes, including: vaping or inhalation. Certain embodiments, provide for nicotine salt containing solution(s) supplied for delivery modes, including: transdermal, oral, inhalation, insufflation, catheterization, or injection. Certain embodiments, provide, for nicotine salt containing solution(s) supplied for delivery modes, including: vaping, inhalation, transdermal, oral, inhalation, insufflation, catheterization, or injection.

Certain embodiments, provide for nicotine salt containing solution(s) supplied for delivery modes, including: vaping, inhalation, transdermal, oral, inhalation, insufflation, catheterization, or injection; wherein the nicotine salts are characterized by a smooth user experience.

Certain embodiments, provide for nicotine salt containing solution(s) supplied for delivery modes, including: vaping, inhalation, transdermal, oral, inhalation, insufflation, catheterization, or injection (and more preferably for vaping, inhalation, oral, or inhalation); wherein the nicotine salts are characterized by a “bite” or “biting” user experience (for example, having a bracing quality to the experience).

The “bite” or “throat hit” can be further modified and altered by the chemistry of the functional groups in the complex, as embodied herein. For example, nicotine hexanoate has no functional groups left unbound to nicotine in the complex. The saturated carbon “tail” possesses little electron density compared to that of nicotine citrate, which possesses two unbound carboxylic acid groups. Therefore, in certain embodiments, nicotine citrate therefore provides a stronger throat hit compared to that of nicotine hexanoate, which provides a “smoother” sensation in the respiratory tract. These results were confirmed by an in-person sampling of electronic cigarette users to evaluate user perception of different nicotine salts, the free electron density, and how this steric hindrance relates or correlates to user experience and satisfaction.

Embodiments nicotine salt solutions disclosed herein, provide a range of pleasurable experiences for the user or patient (if in need of treatment for a condition with an embodied composition. For example, in certain embodiments, a composition of the present invention is for treating or is used by one who seeks nicotine replacement therapy (NRT). In certain embodiments, the electronic cigarette user seeking to remove their mental dependence on the satisfaction achieved from that of a cigarette, has an option to take control over this aspect of nicotine delivery, using the compositions and methods embodied in the present invention. In certain embodiments, this approach provides the NRT or tobacco product formulator with control over user experience variables and is able to improve upon the effectiveness of such products.

In certain embodiments, the experience from using combustion type products, such as cigarettes, is preferred by some nicotine users because they describe a perception of a “throat hit” sensation in their respiratory tract. This experience is associated with pleasure for many tobacco smokers. In traditional e-cigarettes that use purified, free-base nicotine, this “throat hit” experience does not occur. With the nicotine salt-based solutions embodied and described herein, users of vaping (e-Liquid) products and other nicotine replacement therapy solutions, such as oral lozenges, chewing gum, transdermal patches, intranasal sprays, inhalers can obtain various levels of satisfaction using an instant manufacture of the invention that includes one or more nicotine salts complexes. These complexes, whether simple, complex, higher order (heterogeneous), or bridged (embodiments described herein), are used in methods to alter a formulation to deliver nicotine to the user, or patient, in a manner that is conducive to a pleasurable experience. In certain embodiments of having or using a transdermal patch, the user may prefer a faster onset of nicotine, potentiated by a more hydrophilic formulation compared to that of a traditional nicotine formulation being more hydrophobic. For a lozenge or chewing gum, the user may prefer a modified formulation that masks the sharp, harsh sensation of that of freebase nicotine solutions cigarette smokers. For electronic cigarettes, certain vaping solutions disclosed herein, provide a sharp throat hit, allowing for a patient or user to experience the nicotine in a defined, well targeted area of the respiratory tract. Other vaping solutions disclosed herein provide a smooth (without harshness) vaping experience, wherein the user is minimally sensate to the effects of nicotine in the respiratory tract. Still other vaping solutions disclosed herein provide a mixture of smooth and harsh character.

Certain embodiments provide or disclose molecules can form “bridged” nicotine salt complexes. Such organic acids possess two or more carboxylic acid functional groups, separated by between 2-3 carbons in their length chain. In certain embodiments, the chain must be saturated (e.g.: nicotine fumarate is excluded and, for example, nicotine malate can be bridged). Certain embodiments provide bridged nicotine salt complexes, including: nicotine malate (1:1 bridged), nicotine succinate (1:1 bridged), or nicotine tartrate (1:1 bridged) for 2-carbon separation; and nicotine glutarate (1:1 bridged) for 3 carbon separation. The notation embodied herein is unique from a 1:1 complex as the organic acid is bound twice to one molecule of nicotine.

Certain embodiments provide a method for the manufacture a nicotine salt complex that binds two molecules of the same organic acid to both nitrogen atoms of nicotine, the organic acid is quickly added in an acid:nicotine molar ratio greater than 1:1, preferably 1:2. The dissociation of relatively large amounts of the organic acid in solution provides protons for the N-methyl pyrrolidinyl nitrogen, allowing it to form both ionic and hydrogen bonds with the acid. Molar ratios of greater than 1:1 promote further hydrogen bonding between the acid and pyridinyl nitrogen, allowing for higher order nicotine salt complexes.

In certain embodiments, individual higher order or simple nicotine salt complexes can be further combined and mixed into a complex stock solution, containing two, three, four five, or more separate organic acids or one, two, three, four, five, or more different nicotine salt complexes. In certain embodiments, the more complex the solution, i.e. greater the number of total nicotine salts in the solution, the more the nicotine salt stock solution produced as an embodiment of this invention will satisfy the user for a longer period of time before becoming “averse” to the formulation. Users commonly become overly sensate to a specific type or subtype of compounds, and in the case of nicotine salts, will sensate them less than would be desirable. By using multiple combinations of simple and higher order nicotine salt complexes, the formulation can be made more robust and enjoyable for the user for longer periods of time.

In certain preferred embodiments, the nicotine salt-based solutions of the present invention, users of vaping (e-Liquid) products and other nicotine replacement therapy solutions, such as oral lozenges, chewing gum, transdermal patches, intranasal sprays, inhalers obtain selected levels of satisfaction or experience (e.g., throat hit or smooth and degree between) by the manufacture of the solution to include one or more nicotine salts as described herein. In certain embodiments, these complexes, or salts, are utilized to alter a formulation to deliver nicotine to the user, or patient, in a manner that is conducive to a pleasurable experience. For embodiments including or using a transdermal patch, the user may prefer a faster onset of nicotine, potentiated by a more water-soluble formulation compared to that of a traditional nicotine formulation being of more oil solubility. For embodiments including or using a lozenge or chewing gum, the user may prefer a modified formulation that masks the sharp, harsh sensation of that of freebase nicotine solutions. For embodiments including or using an electronic cigarette, certain vaping solutions disclosed herein provide a sharp throat hit, allowing for a patient or user to experience the nicotine in a defined, well target area of the respiratory tract. Other vaping solutions disclosed herein provide a smooth (without harshness) vaping experience, wherein the user is minimally sensate to the effects of nicotine in the respiratory tract. Still other embodiments of vaping solutions disclosed herein provide a mixture of smooth and harsh character. Embodiments having different nicotine salt solutions provide a range of pleasurable experience for the user or patient, who seeks an effective nicotine replacement therapy. In addition, the electronic cigarette user seeking to remove their mental dependence on the satisfaction achieved from that of a cigarette, has an option to take control over this aspect of nicotine delivery through the embodiments of the present invention. Embodiments with this approach allow the NRT user or electronic cigarette formulator to take control over embodied variables, and improve upon the effectiveness of such products.

In certain optional embodiments, the use of nicotine salt complexes and methods therefor, for the manufacture of cigarettes or cigarette tobacco, are specifically disclaimed.

Certain embodiments provide methods to manufacture large quantities of pure liquid nicotine salts from free-base nicotine, with the use of specific reaction parameters and procedures, and equipment.

In certain embodiments, the binding action between nicotine and an organic acid has many desirable and beneficial characteristics for the nicotine user or patient (e.g., an NRT patient), including, but not limited to, the following three methods:

1) Nicotine complexed to an organic acid is more stable and resistant to oxidation in solution, as embodied in certain aspects herein. The bond between the protonated pyrrolidinyl nitrogen and the organic acid (or conjugate base, thereof) hinders oxidation of nicotine at the pyrrolidine center and at the pyridine center.

2) In certain embodiments herein, different nicotine-organic acid salts vary in their character. While free-base nicotine possesses a differing and characteristic “flavor” and “throat sensation” or “throat hit,” nicotine salts offer additional and, in certain embodiments, different user experiences having both pleasing, positive effects. Whether by smoothening, such as reducing, the “throat hit” or throat sensation or by increasing the amount of throat “bite”, specific formulations have desirable characteristics that favorably alter the user's vaping experience compared to free-base nicotine and, in some aspects, depends on the preference(s) of the user. These methods, can be modified, altered, and/or controlled, in certain embodiments for example, by the application of embodied organic acids to bind to certain centers of nicotine in an ordered fashion (i.e., the electronegative centers of the nitrogens in one or both rings of the nicotine). This binding can be controlled by the chemist or manufacturer through an embodiment of the present invention, such as by modifying the pH of the solution using an acid or base, including a strong acid or base such as HCl or NaOH, respectively, to lower or raise the pH of an embodied composition, to achieve an embodied experience or outcome for the user or patient. In certain embodiments, the modifications of the solutions to achieve a selected experience (i.e., a method for making), include: A) to lower the pH to from 4.0 to 6.5, if needed as depends on the equilibrium pH of the selected nicotine salt or combination of multiple salts, and preferably from 5.0 to 6.0; thereby enhancing or providing a nicotine salt solution with a smooth characteristic or attribute (or a reduced “throat hit”) sensation to the user; or B) to raise the pH of a nicotine salt complexed solution, if needed as depends on the equilibrium pH of the selected nicotine salt or the combination of salts to from 6.0 to 8.0, (preferably from 6.0 to 7.0 or 6.1 to 7.0) thereby enhancing or providing a nicotine salt solution with a “throat hit” characteristic or attribute.

In certain embodiments, and due to their character, certain embodied nicotine salts are useful in other nicotine delivery systems besides vaping, for example, but not limited to: lozenges, gums, transdermal patches, intranasal formulations, snuff, snuss, and dip.

3) In certain embodiments, the nicotine-organic acid salt complex is most stable in a pH range near the pKa of the organic acids selected or employed (or pKa's for multi-protic or multi-functional). In certain embodiments, a preferred pH range in near neutral pH (7.0+/−0.75) is preferable for the addition of flavorants, excipients, and solubilizers, especially if starting from an acidic equilibrium before adjusting the pH, if adjusted.

Nicotine in its free-base form is basic at pH˜10 depending on concentration. Nicotine free-base has two free nitrogenous centers with a high amount of electron density, yielding an off-putting harshness, to many users, when introduced to the respiratory tract, or into the oral cavity. In certain embodiments herein, the harshness is determined to be attributable to a free-electron density, or electronegativity, of the free-base nicotine, which attributes are ready to interact with compatible chemical groups in the molecules present in the latent environment (which may vary per delivery mode). When free-base nicotine is perceived by the user in the respiratory tract or oral cavity, the free electron density yields a harsh, off-putting flavor or perception, which is colloquially referred to as “harshness.”

As embodied herein, harshness can be modified or controlled by way of formation of a nicotine salt complex. The complex works to buffer the high pH of free-base nicotine, which yields a more pleasurable or less “harsh” experience in the oral cavity or respiratory tract. The cellular membranes of these specific mucosa are sensitive to changes in pH, of which the nicotine salt allows for a more static pH of the mucosa throughout the absorption process of nicotine. Free base nicotine would lead to an abrupt increase in pH of these mucosa at the points of delivery, leading to, or enhancing an alkaline damaging effect. In embodiments herein, harshness is significantly reduced when replacing free-base nicotine with more or more nicotine salt complex(es). The selection or provision, as described herein, of said constituents yields or results in the formation of embodied nicotine-containing product(s) having a controlled or controllable enhancement to the user experience and is perceived as pleasurable per the preferences of the selected user, having control of the relative “throat hit” or “bite” versus smooth characteristic of the instant nicotine salt complex(es) or solutions; including at the production stage or at the control of a user or a formulator. In certain embodiments, compositions or solutions of the present invention are produced or altered as described and as necessary, to be in a range associated with the mucosa or surface at the point of delivery. For example, preferred compositions having with a pH range of 2.0 (preferably 1.5, more preferably 1.0 and still more preferably 0.5 pH units, above and below a pH range of the mucosa or surface at the point of delivery.

In certain embodiments, biologically suitable carriers (e.g., a liquid solvent) for the nicotine salt complexes described herein include a medium in which the nicotine salt complex is soluble at ambient temperatures, such that the nicotine salt does not form a precipitate, or at least does not form an excessive precipitate. The degree of precipitation, or lack thereof, can be visually determined by the producer, formulator or user having visual access a sample. Examples of suitable carriers, include: but are not limited to, vegetable glycerin/glycerol, propylene glycol, water, and ethanol as well as each combination or premutation thereof. In some embodiments, the liquid carrier comprises 0% to 100% of vegetable glycerin and 100% to 0% propylene glycol. In some embodiments, the liquid carrier comprises 10% to 70% propylene glycol and 90% to 30% vegetable glycerin. In some embodiments, the liquid carrier comprises 20% to 50% of propylene glycol and 80% to 50% vegetable glycerin. In some embodiments, the liquid carrier comprises 30% propylene glycol and 70% vegetable glycerin.

Certain embodiments provide a composition comprising a nicotine salt in a solution for vaping, comprising: a nicotine molecule complexed with an acid to form the nicotine salt, wherein the acid, when not complexed, includes one or more dicarboxylic acids and one or more keto acids forming the salt. In a preferred embodiment the solution has a pH above 6.7, for example above 6.7 and up to 8.0. In alternative embodiments, the pH is from 3.0 to 6.7.

In certain embodiments, an alternate manner of writing a numeral or number with a decimal point in it is in the form of numeral dot numeral. In this alternate manner, a pH of 6.7 can be optionally expressed as 6 dot 7, including in the claims.

Certain embodiments provide a composition comprising a nicotine salt in a solution for vaping, comprising: a nicotine molecule complexed with an acid to form the nicotine salt, wherein the acid, when not complexed, includes one or more monocarboxylic acids and one or more dicarboxylic acids. In a preferred embodiment, the solution has a pH of about 6.0 to 6.3. Other pH values and ranges are optional and can include a pH from 3.0 to 8.0, in one example.

Certain embodiments provide an adjusted pH of a solution containing one or more nicotine salt complexes. The pH can be adjusted using opposing acid or basic solution which can include sodium hydroxide to raise the pH (to make the solution more basic) and hydrochloric acid to lower the pH (to make the solution more acidic), as embodied herein.

Certain embodiments provide a nicotine molecule complexed with an acid. A preferred complex includes one or more hydrogen bonds between the organic acid, or its conjugate base, and the nicotine, without being bound to mechanism.

Certain embodiments provide a solution that is manufactured from a free-base nicotine molecule and from one or more organic acids forming a nicotine salt complex in the solution.

Certain embodiments provide a solution for vaping. Vaping solutions (e-Liquids) are preferably made with a nicotine salt composition disclosed herein and, preferably, use vape solution manufacturing technics disclosed herein, and which may include the incorporation of vegetable glycerin (VG) or propylene glycol, or both into the compositions.

In certain embodiments of the invention, a vaping solution optionally includes flavor and aroma enhancers.

Table 2 provides embodiments of preferred nicotine salt complexes and nicotine:organic acid ratio(s) preferred for the given organic acids and examples of permissible formations of higher order complex(es) (i.e., heterogenous nicotine salt complex formation).

TABLE 2
Nicotine Salt Complexes and Nicotine:Organic
Acid Ratios for Preferred Formations

| Column A2 | Column B2 |
|---|---|
| lactic acid | (1:1 and 1:2) |
| 4-hydroxybenzoic acid | (1:1 and 1:2) |
| propionic acid | (1:1 and 1:2) |
| glycolic acid | (1:1 and 1:2) |
| nicotinic acid | (1:1 and 1:2) |
| formic acid | (1:1 and 1:2) |
| acetic acid | (1:1 and 1:2) |
| benzoic acid | (1:1 and 1:2) |
| valeric acid | (1:1 and 1:2) |
| salicylic acid | (1:1 and 1:2) |
| acetylsalicylic acid | (1:1) |
| oxalic acid | (1:1 and 1:2) |
| malic acid | (1:1, 1:1 bridged, and 1:2) |
| succinic acid | (1:1, 1:1 bridged, and 1:2) |
| tartaric acid | (1:1, 1:1 bridged, and 1:2) |
| fumaric acid | (1:1 and 1:2) |
| levulinic acid | (1:1 and 1:2) |
| pyruvic acid | (1:1 and 1:2) |
| acetoacetic acid | (1:1 and 1:2) |
| citric acid | (1:1 and 1:2) |
| isocitric acid | (1:1 and 1:2) |
| aconitic acid | (1:1 and 1:2) |
| propane-1,2,3-tricarboxylic acid | (1:1 and 1:2) |
| trimesic acid | (1:1 and 1:2) |
| glutaric acid | (1:1, 1:1 bridged, and 1:2) |
| 3-hydroxyglutaric acid | (1:1, 1:1 bridged, and 1:2) |
| Malonic acid | |
| Adipic acid | |

Certain embodiments herein, provide methods for composition manufacture, including, but not limited to: selection of one or more organic acids from Column A of Table 1, one or more organic acids from Column B of Table 1, wherein the paring in Table 1 are representative of embodied methods of selection thereof including by determination of steric hindrance and bonding to the two centers of the nicotine molecule having their characteristic pKa attributes, which is embodied herein. The present invention further embodies methods for manufacture of nicotine salt complexes, including, but not limited to: stoichiometric chemistry profiles of embodied complexes as illustrated in Table 2, Columns A2 and B2.

In certain embodiments, organic acids having fewer than 6 carbon straight length chain (AKA caproate/hexanoate) are determined to form 1:1 single complexes or 1:2 high order salt complexes—example: nicotine dicitrate, dibenzoate, ditartrate, dioxalate. For aromatic or branched molecules, additional determinations of steric hindrance and electronegativity, shape and size are embodied herein. Some branched molecules are embodied for separation of binding sites on higher order salts to allow for 1:2 formations. For example, without limitation: Nicotine dicitrate.

In certain embodiments, higher order (heterogeneous) complexes are formed use specific methods to select binding “pairs,” with examples provided herein, including in Tables 1 and 2. In certain embodiments, the organic acid molecule(s) do not possess (lack) competing functional groups that would repel each other into an unfavorable conformer, the higher order salt are not likely to form or do not form at embodied energies and other attributes. In certain embodiments, both binding pair are sterically compatible by way of their functional groups, the total number and arrangement of carbons, and electrical environment (sigma vs pi bonds leading to enhanced distribution of electron density), the higher order salt are formed as embodied herein. An example herein of a formed higher order salt is nicotine N-malate-N′-benzoate. An example herein of an unlikely formation of a higher order salt is Nicotine N-trimesate-N′-Citrate, which is not formed, or not a significant product herein (without being bound to mechanism, the production of the later example is reduced or eliminated through functional group/steric hindrance).

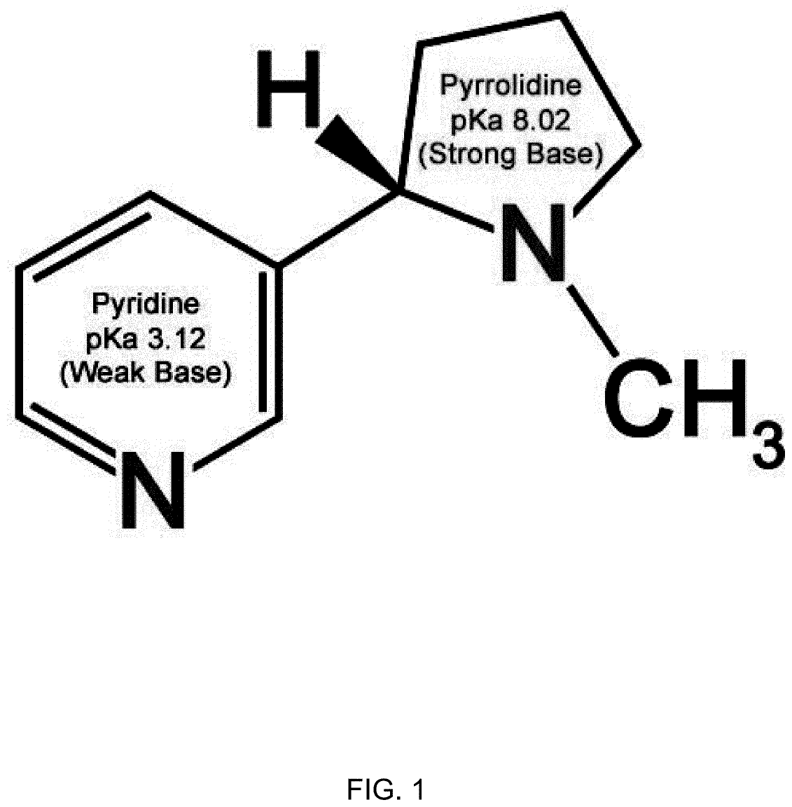

An example of embodied methods of manufacture for bind nicotine to one molar equivalent of oxalic acid at the N-methyl pyrrolidine center, and one salicylic acid molar equivalent to the nitrogen at the pyridinyl center, is nicotine N-salicylate-N′-oxalate. In certain embodiments, oxalic acid is added to nicotine at the preferred pH for the embodiment (in certain embodiments, at the pKa of oxalic acid). In embodiments, the oxalic acid is bound by hydrogen forces (hydrogen binding) to the nicotine at the N-methyl pyrrolidine center by way of conditions described herein (including by way of pH, pH/pKa matching, steric hindrance considerations, molar ratios and other embodied attributes). In certain embodiments, the oxalic acid binding is reacted to equilibrium and, optionally, the pH of the solution is adjusted to that of the pKa of salicylic acid, in the example, which is near pKa 3, resulting in a binding of salicylic acid at the pyridinyl center (preferably as by hydrogen bonding). FIG. 1 depicts a representative nicotine molecule of the present invention, with specific pKa's of 3.12 (pyridine ring) and 8.02 (pyrrolidine ring).

In certain embodiments, oxalic acid, continuing in this example, is added before the salicylic acid, in an order, as the methods herein predict that adding the salicylic acid first (before the oxalic acid) will reduce or eliminate complex formation of the complete complex. For example, the salicylic acid would bind at the N-methyl pyrrolidine center, which would reduce or disallow the oxalic acid to bind at the pyridinyl center, due to the attributes and methods embodied herein, including the attribute of steric hindrance. In certain embodiments, the competitive binding by both organic acids (as in a method of adding both organic acids in together or temporally, if not physically admixed) is predicted by embodiments herein to result in competition for the N-methyl pyrrolidine center by each (both) organic acids and a predicted reduction in heterogenous complex formation. The embodiments set forth in the present example can be extended to other embodiments of compositions and methods herein.

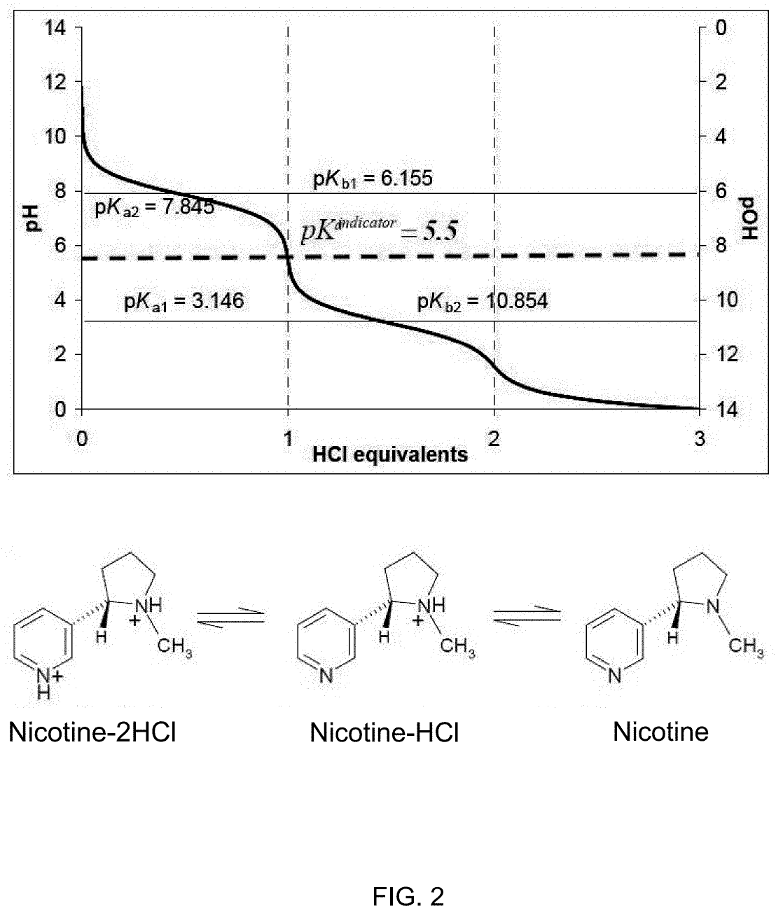

Compounds that possess free electron density within their functional groups aside from the carboxylate bound to nicotine by ionic forces will yield a salt that is stronger in “throat hit.” This “throat hit” can be further modified and altered by the chemistry of the functional groups in the complex. Example: Nicotine hexanoate has no functional groups left unbound to nicotine in the complex nicotine-hexanoate. This keto group possesses very little electron density compared to that of nicotine malate, which leaves a free hydroxyl group, and free carboxyl group in the 1:1 complex. Nicotine citrate therefore provided a stronger throat hit compared to that of nicotine hexanoate which provided a smoother sensation in the respiratory tract. These results were colluded by an in-person sampling of electronic cigarette users to evaluate user perception of different nicotine salts, their free electron density, and how this correlates to user experience and satisfaction. As shown at FIG. 2, the nicotine molecule is altered according to desired pH, which directly corresponds with either acid, neutral or basic conditions.

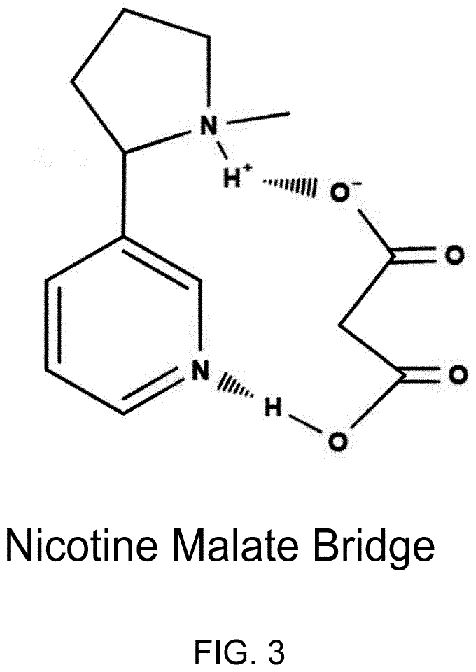

Certain molecules can also form “bridged” complexes, in which the organic acid possesses two or more carboxylic acid functional groups, separated by between 2-3 carbons in their length chain. The length chain must be unsaturated. Examples of successful bridged complexes: Nicotine-Malate (1:1) (see FIG. 3), Nicotine-Succinate (1:1), or Nicotine-Tartrate (1:1) for 2-carbon separation, nicotine glutarate (1:1) for 3 carbon separation.

Once the first hydrogen bond has been formed at the N-methyl pyrrolidine on the nicotine molecule with one of the carboxylic acid functional groups on the organic acid, the mixture is adjusted to the pKa1 of the organic acid in question. This will allow for a deprotonation of the second functional group to potentiate binding with the pyridinyl nitrogen on the nicotine molecule. Example: Malic acid is added to nicotine in a 1:1 ratio at a pH of around 6.5 (pH of nicotine malate). The next step would be to bring the mixture to a pH of 5.03 (pKa1) to allow for the second carboxylic acid functional group to bind. Nicotine malate 1:1 has been formed with a bridge. This novel molecule will possess a “smoother” character when inhaled or exposed to the respiratory tract due to the electron density now being ionically bound to both nitrogenous groups on the nicotine molecule.

Individual higher order or simple nicotine salt complexes can be further combined and mixed into a complex stock solution, containing one, two three, four five, or more separate nicotine salts. The more complex the solution is, i.e. number of total nicotine salts in the solution, the higher likelihood that the nicotine salt stock solution produced as an embodiment of this invention will satisfy the user for a longer period of time before becoming “averse” to the formulation. Users commonly become overly sensated to a specific type or subtype of compounds, and in the case of nicotine salts, will sensate them less than would be desirable. By using multiple combinations of simple and higher order nicotine salt complexes, the formulation can be made more robust and enjoyable for the user for longer periods of time.

### Skin: Transdermal Patches:

Users of transdermal patches seek nicotine through a transdermal delivery system as a nicotine replacement therapy (NRT), commonly used to quit smoking. Users apply a patch to the surface of the skin to diffuse nicotine across the epidermis and into the blood-vessel-containing areas—the dermis and hypodermis—to diffuse nicotine into the bloodstream. The nicotine diffusion across these areas is potentiated by the increased water-solubility of nicotine salt compounds, bridges, and higher order complexes, as well as the pH value of the complex being selected to be within a target pH range of the latent environment of the target delivery site. The faster absorption of a nicotine salt compared to the oil-soluble nicotine free-base allows for diffusion into the dermis and hypodermis with faster pharmacokinetics. Once the nicotine salt is deposited into the dermis, it is solubilized within the interstitial space, and the nicotine is then separated from the organic acid. The acid-base buffering effect allows for a diffusion of nicotine into the capillary tissue without basic shock to the cells exposed, allowing for a faster and more rapid diffusion into the capillaries of the epidermis and hypodermis.

The majority of the human body's skin is at pH 5.5, which is suitable for many formations of complex, higher order, and bridged salts. Within a pH range of 4.5-6.5, a formulation comprising one or more simple, complex, higher order, or bridged nicotine salt complexes is suitable for a transdermal patch. The goal is to be able to deposit nicotine on the surface of the skin at around pH 5.5, which will allow for separation of the nicotine from the complex at the epidermis, then diffuse into the more non-polar layers of the dermis and hypodermis containing the blood capillaries. A higher order nicotine salt such as Nicotine N-malate-N′-oxalate, expressing a pH close to that of 5.5, would be an ideal candidate for such formulation. Another example might be nicotine dicitrate, of which possesses a similar pH in final formulation for transdermal application.

### Oral: Chewing Tobacco Replacements/Lozenges/Chewing Gum:

Oral delivery of nicotine is commonly accomplished through popular nicotine replacement therapy (NRT) methods such as lozenges and chewing gum, as well as herbal-based dips and chews as replacements for traditional chewing tobacco. These products have nicotine added to the formulation in the form of a time-release complex, usually as nicotine polacrilex. However, for the user, a faster onset of nicotine delivery into the capillary beds of the oral cavity (inside cheek or sublingually) may be desired. A formulation comprising a nicotine salt complex would utilize such embodiments to accomplish a faster onset of nicotine satisfaction for the user. The rates of delivery can be controlled as a ratio of nicotine salt complexes to nicotine polacrilex to allow for a fast onset followed by prolonged rate of diffusion of nicotine to the user. The nicotine diffusion across these areas is potentiated by the increased water-solubility of nicotine salt compounds, bridges, and higher order complexes. The faster absorption of select nicotine salt complexes compared to the oil-soluble nicotine free-base or nicotine polacrilex allows for diffusion into the capillary beds of the oral cavity with faster pharmacokinetics. Once the nicotine salt is deposited into the mucosa, it is separated from the organic acid. The acid-base buffering effect allows for a diffusion of nicotine into the capillary tissue without basic shock to the cells exposed, allowing for a faster and more rapid diffusion into the capillaries of the oral cavity.

An embodiment of this invention would be the use of Nicotine Salts Complexes that are uniquely suited for chewing gum, lozenges, or other formulations with intent to deliver nicotine to the user by way of the capillary beds of the oral cavity. The oral cavity has an extremely varied pH, dependent on the foods eaten, rate of salivation, microbial environment, acid reflux, and a multitude of other factors. This area of delivery for nicotine is the most difficult, but possesses a high amount of capillary beds—an ideal candidate for nicotine delivery. A nicotine salt complex must be chosen with a near-neutral pH formulation. The alkaline buffering effect of the organic acid once unbound to nicotine is crucial for resisting alkaline shock to the cells within the oral cavity. This effect will further potentiate nicotine diffusion without shock to the cells exposed. A simple nicotine salt complex such as Nicotine propionate or nicotine acetate, expressing a pH close to that of 6.5 to neutral, and also possessing little to no free electron density outside of the bound carboxylic acid moiety (a monocarboxylic acid), would be an ideal candidate for such formulation. Another example might be a 1:1 “bridged” Nicotine malate, of which possesses a similar pH and has no free electron density outside of the bound carboxylic acid moieties (both carboxylic acid groups are bound to both the pyrrolidinyl and pyridinyl nitrogens on the nicotine molecule. The oral cavity is the most sensitive to tastes and variance in pH—the formulation must be chosen carefully so as to pair an organic acid with non-displeasing flavor. If a compound such as nicotine valerate was chosen, once deposited into the oral cavity, the valerate weak base may be perceived by taste buds as displeasing.

An embodiment of this invention would be the use of Nicotine Salts Complexes that are uniquely suited for chewing tobacco replacements. Nicotine salt complexes with a pH close to neutral, for example, nicotine levulinate (FIG. 4A) combined with “bridged” nicotine malate as a complex mixture, would be applied to a cellulose-based, commonly herbal substrate with, flavored, prepared, and pH balanced to as close to neutral as allowed by the formulation. The addition of nicotine salt complexes to this type of formulation would assist in the faster time to nicotine saturation in the capillary beds of the oral cavity, leading to nicotine satisfaction more quickly than that of standard free-base nicotine of a higher pH value.

### Nasal: Intranasal Spray/Snuss:

Nasal sprays are nicotine replacement therapy devices that are commonly formulated with free-base nicotine to deliver a quantity of nicotine to the patient. These formulations are intended to give a relief to the patient for nicotine addiction, and functions by depositing nicotine on the nasal mucosa. These products have been reviewed as very irritating, due to the alkaline shock of free-base nicotine on the cells of the nasal mucosa.

An embodiment of this invention would be the use of Nicotine Salts Complexes that are uniquely suited for nasal spray inhalers. The nasal mucosa, being of pH 5.5-6.5 in a healthy adult, is suited for nicotine salt compounds, bridges, and higher order complexes. Once the nicotine salt is deposited into the mucosa, it is separated from the organic acid. The acid-base buffering effect allows for a diffusion of nicotine into the capillary tissue without basic shock to the cells exposed, allowing for a faster and more rapid diffusion into the capillaries of the nasal cavity.

An embodiment of this invention would be the use of Nicotine Salts Complexes that are uniquely suited for snuff. A pulverized herbal formulation or other carrier cellulose-based substrate is used as a replacement for traditional pulverized tobacco in snuss. Nicotine salt complexes with a pH close to neutral, for example, nicotine levulinate combined with “bridged” nicotine malate as a complex mixture, would be applied to a cellulose-based, commonly herbal substrate with, flavored, prepared, and pH balanced to as close to neutral as allowed by the formulation. The addition of nicotine salt complexes to this type of formulation would assist in the faster time to nicotine saturation in the capillary beds of the oral cavity, leading to nicotine satisfaction more quickly than that of standard free-base nicotine of a higher pH value.

Oral delivery of nicotine is commonly accomplished through popular nicotine replacement therapy (NRT) methods such as lozenges and chewing gum, as well as herbal-based dips and chews as replacements for traditional chewing tobacco. These products have nicotine added to the formulation in the form of a time-release complex, usually as nicotine polacrilex. However, for the user, a faster onset of nicotine delivery into the capillary beds of the oral cavity (inside cheek or sublingually) may be desired. A formulation comprising a nicotine salt complex would utilize such embodiments to accomplish a faster onset of nicotine satisfaction for the user. The rates of delivery can be controlled as a ratio of nicotine salt complexes to nicotine polacrilex to allow for a fast onset followed by prolonged rate of diffusion of nicotine to the user. The nicotine diffusion across these areas is potentiated by the increased water-solubility of nicotine salt compounds, bridges, and higher order complexes. The faster absorption of a nicotine salt compared to the oil-soluble nicotine free-base or nicotine polacrilex allows for diffusion into the capillary beds of the oral cavity with faster pharmacokinetics. Once the nicotine salt is deposited into the mucosa, it is separated from the organic acid. The acid-base buffering effect allows for a diffusion of nicotine into the capillary tissue without basic shock to the cells exposed, allowing for a faster and more rapid diffusion into the capillaries of the oral cavity.

The oral cavity has an extremely varied pH, dependent on the foods eaten, rate of salivation, microbial environment, acid reflux, and a multitude of other factors. This area of delivery for nicotine is the most difficult, but possesses a high amount of capillary beds—an ideal candidate for nicotine delivery. A nicotine salt complex must be chosen with a near-neutral pH formulation. The alkaline buffering effect of the organic acid once unbound to nicotine is crucial for resisting alkaline shock to the cells within the oral cavity. This effect will further potentiate nicotine diffusion without shock to the cells exposed. A simple nicotine salt complex such as Nicotine propionate or nicotine acetate, expressing a pH close to that of 6.5 to neutral, and also possessing little to no free electron density outside of the bound carboxylic acid moiety (a monocarboxylic acid), would be an ideal candidate for such formulation. Another example might be a 1:1 “bridged” nicotine malate, of which possesses a similar pH and has no free electron density outside of the bound carboxylic acid moieties (both carboxylic acid groups are bound to both the pyrrolidinyl and pyridinyl nitrogens on the nicotine molecule. The oral cavity is the most sensitive to tastes and variance in pH—the formulation must be chosen carefully so as to pair an organic acid with non-displeasing flavor. If a compound such as nicotine valerate was chosen, once deposited into the oral cavity, the valerate weak base may be perceived by taste buds as displeasing.

### Cigarettes:

Traditional tobacco cigarettes utilize the form of combustion to atomize and deliver nicotine and other tobacco constituents, of which the Hoffman analytes are classified. These harmful or potentially harmful constituents (HPHCs) are evolved by way of combustion at higher temperatures, usually defined as temperatures greater than 1000 degrees Celsius. At these temperatures, the nicotine salts are fully separated into acid and nicotine components.

Pyrrolization or combustion are detrimental to the delivery methods potentiated by specific formulations of nicotine salt complexes, higher order nicotine salt complexes, and bridged nicotine salt complexes, as their efficacies are partially related to the delivery of the nicotine molecule while bound in its atomized, vaporized, or flashed state. The nicotine salt complex is intended to separate into acid and base components upon deposition to the target membrane, not beforehand.

The activation energy supplied by combustion at temperatures northwards of 1000 degrees Celsius is enough to separate the nicotine complex from the organic acid component and furthermore may oxidize the nicotine at the N-methyl pyrrolidinyl and N-pyridinyl nitrogen centers (of first order is the N-methyl pyrrolidinyl nitrogen center and of second order is the N-pyridinyl nitrogen center). This would alter the solubility, efficacy, and pharmacokinetics of the nicotine deposition onto the target membrane, which is not of interest to this invention. While low temperature combustion is possible, this is also not of interest to the invention as the possibility of the evolution of HPHCs is still of concern. Nicotine salt complexes of claim in this invention are purposed for no heat (oral, transdermal, intranasal, MDIs), low heat (atomization, vaporization), and/or heat-not-burn (atomization, vaporization) technologies.

### Inhaled: Electronic Cigarettes, MDIs—Non-Combustion:

The respiratory tract is an environment extremely varied pH, dependent on the foods eaten, rate of salivation, microbial environment, acid reflux, and a multitude of other factors. This area of delivery for nicotine is the most difficult, but possesses a high amount of capillary beds in a multitude of areas, namely the alveolar mucosa—an ideal candidate for nicotine delivery. A nicotine salt complex must be chosen selectively to consider two major factors, of which are embodiments of this invention: (1) The type of nicotine salt complex(es) and (2) the pH of the overall combination of nicotine salt complexes in the formulation to sensate the user differently. This invention would constitute unique embodiment(s) of the use of Nicotine Salts Complexes uniquely suited for electronic cigarettes and (un)metered dose inhalers (MDIs).

(1) The type of nicotine salt complexes chosen can have an effect on the overall effectiveness of depositing nicotine onto the mucosal membranes of the alveoli in the lungs. A nicotine salt complex between pH 6-7 is preferred for this delivery to delivery to the lungs, as is closest to the target environment of the alveolar mucosa. The alkaline buffering effect of the organic acid once unbound to nicotine is crucial for resisting alkaline shock to the cells within the respiratory tract. This effect will further potentiate nicotine diffusion without shock to the cells exposed on the mucosa, leading to a more efficient diffusion of nicotine into the surrounding capillaries. A nicotine salt complex between pH 5-6 would alternatively be partially deposited in both the alveolar lung mucosa, but also along the pharynx in the upper respiratory tract. A selection of nicotine salts complexes with higher acidic character would be preferred for this application.

(2) The sensation to the user can also be controlled by way of the pH of the overall combination of nicotine salt complexes in the formulation to sensate the user. The inclusion of low pH (5-6) and/or both low pH (5-6) and medium pH (6-7) formulations in the final mixture would be able to both efficiently deposit nicotine onto the alveolar mucosa, and sensate the pharynx, colloquially referred to as “throat-hit,” which can be a pleasing aspect of nicotine inhalation to a majority of users. This is an aspect of the combustion of traditional tobacco cigarettes that can be emulated by the embodiment of this invention for electronic cigarettes, MDIs, and other heat-not-burn technologies. An example of a nicotine salt that would accomplish this task would be a combination of nicotine fumarate, nicotine succinate, and nicotine levulinate. The novel combination of these three salts would accomplish a “throat-hit” sensation from the free electron density provided from the fumarate and succinate unbound secondary carboxylic acid moieties (the first two being hydrogen bound to the pyrrolidinyl nitrogens on the two respective nicotine molecules). At the same time, the nicotine levulinate electron density, being largely consumed by the hydrogen bond to nicotine at the pyrrolidinyl nitrogen, would provide a low-sensation delivery of nicotine to the alveolar mucosa of the lungs. This novel combination of a wide range of pH nicotine salt complex formulations is preferred for embodiments such as electronic cigarettes and (un)metered dose inhalers (MDIs) whose users require both efficient nicotine delivery, and overall positive associations with traditional tobacco cigarettes (“throat hit”).

In antithesis to the “throat-hit,” a nicotine salt complex can be uniquely selected to bypass the upper respiratory tract and deposit directly into the surface of the alveolar mucosa. These formulations are referred to as “smooth.” A nicotine salt complex that possesses a pH of only medium polarity formulations (6-7) is ideal for formulations with a “smoother” character, compared to other organic acids, and compared to free-base nicotine of much higher pH. An example of a nicotine salt that would accomplish this task would be a combination of nicotine levulinate and “bridged” nicotine-malate. The novel combination of these two salts would accomplish a varied, rounded, full-bodied, but “smooth” characteristic from the absence of electron density on the organic acid groups, being largely consumed by the hydrogen bond to nicotine at the pyrrolidinyl nitrogen. This would provide a low-sensation delivery of nicotine to the alveolar mucosa of the lungs. This novel selection of a nicotine salt complex for these formulations are preferred for embodiments such as electronic cigarettes and (un)metered dose inhalers (MDIs).

### Compositions Including Nicotine Salts:

Certain embodiments of the present invention provide a composition, comprising: a concentrated solution including nicotine and one or more organic acids and nicotine salt complexes formed thereof. Certain embodiments provide a composition, comprising: a concentrated solution (e.g., a stock solution) including one or more nicotine salts.

In certain embodiments, preferred organic acids for partnering in a nicotine salt, are selected from the group consisting of: lactic acid, 4-hydroxybenzoic acid, propionic acid, glycolic acid, nicotinic acid, formic acid, acetic acid, benzoic acid, valeric acid, salicylic acid, oxalic acid, malic acid, succinic acid, tartaric acid, fumaric acid, levulinic acid, pyruvic acid, acetoacetic acid, citric acid, isocitric acid, aconitic acid, propane-1,2,3,-tricarboxylic acid or trimesic acid.

In certain embodiments, the higher the pH of an embodied solution, the more dissociated, or ionic, is the character of the solution and there are more free nicotine and organic acid molecules present. In certain embodiments, the lower the pH, the more associated is the character of the solution resulting in more nicotine salt complexes. In certain embodiments herein, when the pH of an embodied nicotine salt containing solution is at the pKa of the organic acid, then essentially 50% of the components are complexed.

Certain embodiments provide composition(s) of a nicotine salt complex or a solution thereof, comprising: a nicotine molecule complexed with an organic acid thereby forming the nicotine salt complex(es). In certain embodiments, wherein the organic acid, includes zero to one or more dicarboxylic acids and one or more keto organic acids.

In certain preferred embodiments the solution has a pH above 6.7, preferably above 6.7 to 8.0 and more preferably 7.0 to 8.0. In certain embodiments, the pH range is from 3.0 to 8.0. In certain embodiments, the pH range is expressed as: 2.5 to 8.5, 3.0 to 8.0 (more preferably), from 3.0 to 8.5 or from 2.5 to 8.0. Certain embodiments provide pH ranges of (from and to): 2.5 to 2.9, 3.0 to 3.9, 4.0 to 4.9, 5.0 to 5.9, 6.0 to 6.9 or 7.0 to 8.0. Certain embodiments provide pH ranges of (from and to): 3.0 to 4.5, 4.0 to 5.5, 4.5 to 5.5, 5.0 to 6.5, 5.5 to 6.5, 6.0 to 6.5, 6.5 to 7.5, 7.0 to 8.5, or 7.5 to 8.5. Certain embodiments provide pH ranges of: 3.0 to 4.0, 4.0 to 5.0, 5.0 to 6.0, 6.0 to 7.0 or 7.0 to 8.0. Certain embodiments provide pH ranges of (from and to): 3.0 to 5.0, 4.0 to 6.0, 5.0 to 7.0, or 6.0 to 8.0. Certain embodiments provide pH ranges of (from and to): 3.5 to 4.5, 3.5 to 5.5, 3.5 to 6.5 or 3.5 to 7.5. Certain embodiments provide pH ranges of (from and to): 3.5 to 5.5, 4.5 to 6.5 or 5.5 to 7.5. Certain embodiments provide pH ranges of: 2.5 to 2.9, 3.0 to 3.9, 4.0 to 4.9, 5.0 to 5.9, 6.0 to 6.9 and 7.0 to 8.0. Certain embodiments provide pH ranges of (from and to): 3.0 to 4.5, 4.5 to 5.5, 5.5 to 6.5, 6.5 to 7.5 and 7.5 to 8.0. The pH values and ranges are useful, in certain embodiments, including for heterogenous nicotine salt complex(es), homogeneous nicotine salt complex(es), monoconjugate nicotine salt complex(es), or other nicotine salt complex(es).

It is understood in the claims that an alternate manner of writing a numeral or number with a decimal point in it is in the form of numeral dot numeral. In this alternate notation, the pH of 6.7 value can be expressed as 6 dot 7.

In certain embodiments, the pH of a nicotine containing solution embodied herein, has a pH resulting from the combination of the nicotine and the organic acid forming nicotine salt complexes, which process comes to an equilibrium effected by the pH of the solution. In certain embodiments, the pH of a nicotine containing solution embodied herein, is adjusted to a desired pH level using a strong acid (e.g., HCl) or base (e.g., NaOH) to a pH value or range embodied herein.

In certain embodiments, the pH of a nicotine salt solution of the present invention is in a range of (from and to): 3.0 to 8.0 or from 2.5 to 8.0. Certain embodiments provide pH ranges of: 2.5 to 2.9, 3.0 to 3.9, 4.0 to 4.9, 5.0 to 5.9, 6.0 to 6.9 and 7.0 to 8.0. Certain embodiments provide pH ranges of (from and to): 3.0 to 4.5, 4.5 to 5.5, 5.5 to 6.5, 6.5 to 7.5 and 7.5 to 8.0. In certain embodiments, the pH or the adjustment of the pH determines the equilibrium of nicotine salt complex formation from the nicotine and the organic acid(s), in the solution, with lower pH values favoring a shift in the equilibrium toward association into nicotine salt(s) and higher pH values favoring dissociation into the respective ions.

In certain embodiments, the selection of organic acids(s) to include in a nicotine salt composition is informed by the pKa values of the organic acid(s),In certain embodiments, a composition of the present invention includes a concentrated nicotine salt solution including nicotine and one or more organic acids or one or more nicotine salt complexes.

In certain embodiments, a composition of the present invention includes a concentrated nicotine salt solution including nicotine and two or more organic acids or two or more nicotine salt complexes.

In certain embodiments, a composition of the present invention includes a concentrated nicotine salt solution including nicotine and three or more organic acids or three or more nicotine salt complexes.

In certain embodiments, a composition of the present invention includes a concentrated nicotine salt solution including nicotine and four or more organic acids or four or more nicotine salt complexes.

In certain embodiments, a composition of the present invention includes a concentrated nicotine salt solution including nicotine and five or more organic acids or five or more nicotine salt complexes.

In certain embodiments, a composition of the present invention includes a concentrated nicotine salt solution including nicotine and six or more organic acids or six or more nicotine salt complexes.

In certain embodiments, the total number of organic acids nicotine salts in an embodied nicotine salt solution is limited to: 1, 2, 3, 4, 5, 6 7, 8, 9, 10, 11, 12, 13, 14, 15, 16, 17, 18, 19, or 20 added organic acids.

In certain embodiments, the number of different nicotine salts complexes formed in an embodied reaction herein, is limited to: 1, 2, 3, 4, 5, 6 7, 8, 9, 10, 11, 12, 13, 14, 15, 16, 17, 18, 19, or 20 nicotine salt complexes; which limited formation can be effected by limiting the reaction time, heated reaction time, mixing time, heated mixing time and the order of reactant addition; for example the order the nicotine, and two or more organic acids are combined for reaction.

### Mixing of Types:

In certain embodiments, a solution of nicotine salts is provided wherein the nicotine salts include: an aromatic acid, a dicarboxylic acid, and a gamma-keto acid. In certain embodiments, use of a nicotine salt(s) based solutions provided herein for vaping (e-Liquid) products by a user results in users reporting, depending on the nicotine salt composition: a harsh characteristic, especially in the area of the throat (internally) which is often referred to as “throat hit,” a smooth vaping experience (having a lack of throat hit) and a mixed experience having a smooth characteristic with a hint of throat hit. Embodied vaping solutions provide a range of pleasurable experience for the vaping enthusiast. Certain embodiments provide a composition, comprising: a concentrated solution including one or more nicotine salts. As used herein, a concentrated solution of one or more nicotine salts refers nicotine salts with the concentration(s) in the ranges shown in Table 3.

TABLE 3
Concentrated Nicotine Salt Solutions
Concentration Ranges of One or More Nicotine Salts Herein
(total for solutions including more than one nicotine salt, in mg/ml)

| Low end of range | High end of range |
|---|---|
| 60 | 750 |
| 70 | 750 |
| 75 | 750 |
| 100 | 750 |
| 120 | 750 |
| 200 | 750 |
| 250 | 750 |
| 300 | 750 |
| 400 | 750 |
| 500 | 750 |
| 600 | 750 |
| 60 | 600 |
| 75 | 600 |
| 100 | 600 |
| 120 | 600 |
| 200 | 600 |
| 250 | 600 |
| 300 | 600 |
| 400 | 600 |
| 500 | 600 |
| 60 | 500 |
| 75 | 500 |
| 100 | 500 |
| 120 | 500 |
| 200 | 500 |
| 250 | 500 |
| 300 | 500 |
| 400 | 500 |

Certain embodiments provide a composition comprising a nicotine salt in a solution for vaping, comprising: a nicotine molecule complexed with an acid thereby forming a nicotine salt, wherein the acid, when not complexed, includes one or more monocarboxylic acids and one or more dicarboxylic acids. In a preferred embodiment, the solution has a pH of about 6.0 to 6.3 and other embodied pH ranges are provided herein, above. Certain embodiments of the present invention provide a solution, comprising: a nicotine salt complex selected from an embodiment disclosed herein.

By way of understanding, in certain embodiments, a complex of a nicotine salt can be written as nicotine+organic acid↔nicotine conjugate base (of the organic acid), which may, or may not be at equilibrium depending on the conditions which can include, but are not limited to: temperature and temperature shifts, time of reaction and/or mixing (with or without heat), pH and changes of pH including by addition of an inorganic acid (e.g., HCl) or base (e.g., NaOH) or additional organic acids, the pKa's of the organic acid(s), the relative pH value of a solution in comparison to a pKa of one or more constituent (for example: nicotine, the organic acid (or multiple organic acids) present and other reactants, diluents, carriers, etc.),and other factors.

In certain embodiments, a nicotine salt complex is referenced using the following terminology: nicotine (name of a conjugate base of an organic acid), for example, nicotine malate.

In certain embodiments, a nicotine salt complex is referenced using the terminology: nicotine (the name of an organic acid or a list of organic acid names), for example, ( . . . in certain embodiments, a nicotine salt complex includes nicotine and an organic acid, such as malic acid, wherein the nicotine is conjugated with the organic acid). In certain embodiments, reference to nicotine and one or more organic acids as nicotine salt complex(es) is an example of an understanding of the invention, in certain embodiments, that nicotine and organic acids can interact dynamically and can reach an equilibrium, and in either case, deprotonation of one or more organic acid(s) and occur with hydrogen bonding between the nicotine and conjugate base(s) of the one or more organic acids, preferably at one or both nitrogen center of the nicotine molecule(s) (without necessarily being bound to mechanism in all instances).

TABLE 4
Certain Embodiments: Inclusion One
or More Aromatic Organic Acid(s)

Nicotine benzoate (benzoic acid)
Nicotine nicotinate (nicotinic acid)
Nicotine trimesate (trimesic acid)
Nicotine salicylate (salicylic acid)
Nicotine vanillate (vanillic acid)
Nicotine cinnamate (cinnamic acid)
Nicotine 4-hydroxybenzoate (4-hydroxybenzoic acid)
Nicotine acetylsalicylate (acetylsalicylic acid)
Nicotine gallate (gallic acid)

Selected embodiments of nicotine salt complexes are disclosed in Table 5 with selected attributes, thereof.

TABLE 5
Selected Embodiments of Organic Acids and Nicotine Salts
Nicotine Salts with Monocarboxylic Acids

| Monocarboxylic Acid | pKₐ | MW | 2D Diagram of Structure | Nicotine Salt |
|---|---|---|---|---|
| Formic Acid | 3.75 | 46.03 | 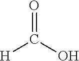 | Nicotine formate |
| Acetic Acid | 4.76 | 60.05 | 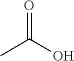 | Nicotine acetate |
| Propionic Acid | 4.88 | 74.08 | 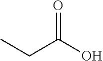 | Nicotine propionate |
| Glycolic Acid | 3.83 | 76.05 | 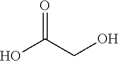 | Nicotine glycolate |
| Pyruvic Acid | 2.50 | 88.06 | 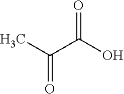 | Nicotine pyruvate |
| Lactic Acid | 3.86 | 90.08 | 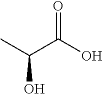 | Nicotine lactate |
| 3-Oxobutanoic Acid (Acetoacetic Acid) | 3.58 | 102.09 | 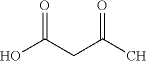 | Nicotine acetoacetate |
| Valeric Acid | 4.84 | 102.13 | 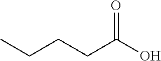 | Nicotine valerate |

Nicotine Salts with Aromatic Acids

| Aromatic Carboxylic Acid | pKₐ | MW | 2D Diagram of Structure | Nicotine Salt |
|---|---|---|---|---|
| Benzoic Acid | 4.19 | 122.12 | 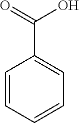 | Nicotine benzoate |
| Salicylic Acid | 2.97 | 138.12 | 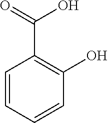 | Nicotine salicylate |
| 4-Hydroxybenzoic Acid (para-hydroxybenzoic acid) | 4.54 | 138.12 | 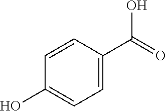 | Nicotine para- hydroxybenzoate |
| Trimesic Acid | 3.12 3.89 4.70 | 210.14 | 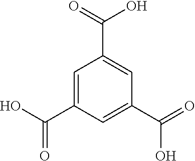 [SMILES: O=C(O)c1cc(C(=O)O)cc(C(=O)O)c1] | Nicotine trimesate |
| Nicotinic Acid | 4.75 | 123.11 | 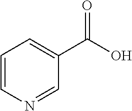 | Nicotine nicotinate |

Nicotine Salts with Dicarboxylic Acids

| Dicarboxylic Acid | pKₐ | MW | 2D Diagram of Structure | Nicotine Salt |
|---|---|---|---|---|
| Oxalic Acid | 1.46 4.40 | 90.03 | 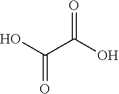 | Nicotine oxalate |
| Malic Acid | 3.51 5.03 | 134.09 | 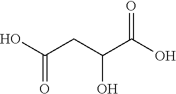 | Nicotine malate |
| Succinic Acid | 4.21 5.63 | 108.09 | 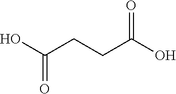 | Nicotine succinate |
| Tartaric Acid | 3.22 4.85 | 150.09 | 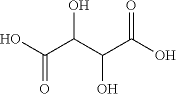 | Nicotine tartarate |
| Fumaric Acid | 3.03 4.54 | 116.07 | 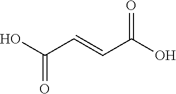 | Nicotine fumarate |

Nicotine Salts with Tricarboxylic Acids

| Tricarboxylic Acid | pKₐ | MW | 2D Diagram of Structure | Nicotine Salt |
|---|---|---|---|---|
| Citric Acid | 3.13 4.76 6.39 | 192.12 | 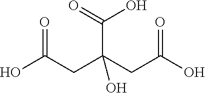 [SMILES: O=C(O)CC(O)(CC(=O)O)C(=O)O] | Nicotine citrate |
| Isocitric Acid | 3.29 4.71 6.40 | 192.12 | 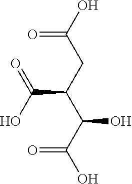 [SMILES: O=C(O)C[C@H](C(=O)O)[C@@H](O)C(=O)O] | Nicotine isocitrate |
| cis-Aconitic Acid | 1.95 | 174.11 | 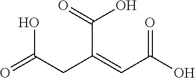 [SMILES: O=C(O)/C=C(/CC(=O)O)C(=O)O] | Nicotine cis-aconitate |
| trans-Aconitic Acid | 2.80 4.46 | 174.11 | 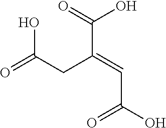 [SMILES: O=C(O)/C=C(\CC(=O)O)C(=O)O] | Nicotine trans-aconitate |

Nicotine Salts with Ketocarboxylic Acids

| Keto Acid | pKₐ | MW | 2D Diagram of Structure | Nicotine Salt |
|---|---|---|---|---|
| Levulinic Acid | 4.64 | 116.11 | 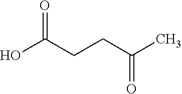 | Nicotine levulinate |

TABLE 6
Selected Embodiments of Organic
Acids and Nicotine Salt Complexes

| Nicotine Salt | |
|---|---|
| Complex | Comment |
| Simple (or Mono) | Nicotine + one organic acid conjugated to the |
| Nicotine Salt | pyrrolidine ring nitrogen center |
| Mixed Simple | A mixture of simple nicotine salt complexes |
| (Mono) Nicotine | |
| Salts | |
| Homogeneous | Nicotine + one organic acid, wherein each |
| Nicotine Salt | nicotine molecule is conjugated to one type of |
| | organic acid molecule, one at each nitrogen |
| | center |
| Mixed Homogeneous | A mixture of homogeneous nicotine salt |
| Nicotine Salts | complexes |
| Heterogeneous | Nicotine + more than one type of organic acid |
| Nicotine Salt | independently conjugated to both nitrogen |
| | centers |
| Mixed | A mixture of heterogeneous nicotine salt |
| Heterogeneous | complexes |
| Nicotine Salts | |
| Bridged | A nicotine molecule + one organic acid |
| Nicotine Salts | bonded to each of the nitrogen centers of the |
| | nicotine in one linkage - bridging the two |
| | nitrogen centers of the nicotine |
| Mixed Bridged | A mixture of bridged nicotine salt complexes |
| Nicotine Salts | |
| Mixtures of the | Mixtures of groupings and of all types. |
| combination or | |
| permutation of | |
| the complexes in | |
| the present | |
| table, above | |

TABLE 7
Selected Embodiments of Nicotine Salt Complexes
and Their Corresponding pKa Value(s)

| Nicotine Salt (Organic Acid) | pKa |
|---|---|
| Nicotine 2-hydroxyisocaproate (2- | 4.26 |
| hydroxyisocaproic acid) | |
| Nicotine 3-hydroxyglutarate (3- | 3.52 |
| hydroxyglutaric acid) | |
| Nicotine 4-hydroxybenzoate (4- | 4.54 |
| hydroxybenzoic acid) | |
| Nicotine acetate (acetic acid) | 4.76 |
| Nicotine acetoacetate (acetoacetic acid) | 3.58 |
| Nicotine acetylsalicylate (acetylsalicylic acid) | 3.49 |
| Nicotine adipate (adipic acid) | 4.43; 5.41 |
| Nicotine alanate (alanine) | 2.34 |
| Nicotine arginate (arginine) | 2.17 |
| Nicotine asparaginate (asparagine) | 2.02 |
| Nicotine aspartate (aspartic acid) | 1.88 |
| Nicotine benzoate (benzoic acid) | 4.19 |
| Nicotine cinnamate (cinnamic acid) | 4.44 |
| Nicotine cis-aconitate (cis-aconitic acid) | 1.95 |
| Nicotine citrate (citric acid) | 2.79 |
| Nicotine cysteinate (cysteine) | 1.96 |
| Nicotine formate (formic acid) | 3.75 |
| Nicotine fumarate (fumaric acid) | 3.03; 4.54 |
| Nicotine gallate (gallic acid) | 4.4 |
| Nicotine gluconate (gluconic acid) | 3.86 |
| Nicotine glutamate (glutamic acid) | 2.19 |
| Nicotine glutarate (glutaric acid) | 4.34 |
| Nicotine glycinate (glycine) | 2.34 |
| Nicotine glycolate (glycolic acid) | 3.83 |
| Nicotine hexanoate (hexanoic acid) | 4.88 |
| Nicotine histidinate (histidine) | 1.82 |
| Nicotine isocaproate (isocaproic acid) | 5.09 |
| Nicotine isocitrate (isocitric acid) | 3.29; 4.71; 6.40 |
| Nicotine isoleucinate (isoleucine) | 2.36 |
| Nicotine isovalerate (isovaleric acid) | 4.77 |
| Nicotine lactate (lactic acid) | 3.58 |
| Nicotine leucinate (leucine) | 2.36 |
| Nicotine levulinate (levuliniuc acid) | 4.64 |
| Nicotine lysinate (lysine) | 2.18 |
| Nicotine malate (malic acid) | 3.51; 5.03 |
| Nicotine malonate (malonic acid) | 2.85 |
| Nicotine methioninate (methionine) | 2.28 |
| Nicotine nicotinate (nicotinic acid) | 4.75 |
| Nicotine oxalate (oxalic acid) | 1.46; 4.40 |
| Nicotine phenylalanate (phenylalanine) | 1.83 |
| Nicotine phthalate (phthalic acid) | 2.76; 4.92 |
| Nicotine prolinate (proline) | 1.99 |
| Nicotine propiolate (propiolic acid) | 1.84 |
| Nicotine propionate (propionic acid) | 4.88 |
| Nicotine pyruvate (pyruvic acid) | 2.50 |
| Nicotine salicylate (salicylic acid) | 2.97 |
| Nicotine serinate (serine) | 2.21 |
| Nicotine succinate (succinic acid) | 4.21; 5.63 |
| Nicotine tartrate (tartaric acid) | 3.22; 4.85 |
| Nicotine threoninate (threonine) | 2.09 |
| Nicotine trans-aconitate (trans-aconitic acid) | 2.80; 4.46 |
| Nicotine trimesate (trimesic acid) | 3.12; 3.89; 4.70 |
| Nicotine tryptophanate (tryptophan) | 2.83 |
| Nicotine tyrosinate (tyrosine) | 2.2 |
| Nicotine valerate (valeric acid) | 4.84 |
| Nicotine valinate (valine) | 2.32 |
| Nicotine vanillate (vanillic acid) | 4.51 |

### EXAMPLES

### Example I

### Identification of Preferred Keto Acid-Nicotine Salt Complexes

It was determined, through experimentation, which of those embodiments were more preferable to others with respect to the keto acid-nicotine salt complexes described in the present invention. Complexes were arranged in descending ranked order based on the following criteria:
(a) Bridging and higher order binding: Preferably, one carboxylic acid functional group resides on one of the termini of the molecule, with at least one keto functional group on another. Bridged keto acid nicotine salt complexes would require a separation of between 2-3 carbons between the keto and carboxylic acid functional groups for bridging to occur.
(b) Steric hindrance: An excess of molecular size is not ideal for preferred embodiments of the present invention.
(c) Quantity of carbons in relation to functional groups: The lower the amount of carbons and the lower amount of excess functional groups on the molecule (other than the required keto- and -carboxylate functional groups)—the more preferred the embodiment. Nicotine pyruvate would not be a likely candidate to form a bridge because it's separation between moieties is less than 2 carbons. Nicotine levulinate, on the other hand, would be a preferred embodiment for ease of forming a bridged complex. 2, 3, and 4-oxo acids are preferable to form bridges with the least amount of chemical groups other than the bound carboxylate and possibly keto functional groups.

Table 8 lists the most preferred keto acid-nicotine salt complexes in order from most preferred to least preferred.

TABLE 8
Preferred Keto Acid-Nicotine Salt Complexes

Nicotine levulinate (levuliniuc acid)
Nicotine oxaloacetate (oxaloacetic acid)
Nicotine mesoxalate (mesoxalic acid)
Nicotine beta-ketoglutarate (beta-ketoglutaric acid)
Nicotine alpha-ketoglutarate (alpha-ketoglutaric acid)
Nicotine pyruvate (pyruvic acid)
Nicotine hydroxypyruvate (hydroxypyruvic acid)
Nicotine 3-mercaptopyruvate (3-mercaptopyruvic acid)
Nicotine 4-hydroxy-2-oxopentanoate (4-hydroxy-2-oxopentanoic acid)
Nicotine 4-hydroxy phenylpyruvate (4-hydroxy phenylpyruvic acid)

### Example II

### Identification of Preferred Aromatic Organic Acid-Nicotine Salt Complexes

It was determined, through experimentation, which of those embodiments were more preferable to others with respect to the aromatic organic acid-nicotine salt complexes described in the present invention. Complexes were arranged in descending ranked order based on the following criteria:
(a) Auxiliary (free) functional groups: Molecules with the least amount of unbound carboxylate, alcohol or ester functional groups (other than those bound to nitrogens on the nicotine molecule) are the most preferred embodiments.
(b) Steric hindrance: An excess of molecular size is not preferable for embodiments described under this aromatic organic acid-nicotine salt complex.
(c) Possibility of forming higher order salts or bridges is preferred: An organic acid that possesses two or more carboxylic acids and or keto functional groups are preferred embodiments for the formation of a bridged complex if separated by 2-3 carbons between the carboxylic acid functional groups.

Analysis revealed nicotine benzoate would be the most preferred embodiment of this invention when classified by the principle of free electron density. It is the simplest aromatic organic acid, followed by salicylic acid, and similarly, 4-hydroxybenzoic acid. Nicotine benzoate would also be the most preferred embodiment when arranging by the principle of steric hinderance. Benzoate hydrogen bound to the pyrrolidine nitrogen would allow for the most relaxed bond angles when binding secondary organic acids to the pyridinyl nitrogen. For the same reason, nicotine benzoate would be the most preferred embodiment regarding the principle of higher order complexes.

Table 9 lists the most preferred aromatic organic acid-nicotine salt complexes in order from most preferred to least preferred.

TABLE 9
Preferred Aromatic Organic Acid-Nicotine Salt Complexes

Nicotine benzoate (benzoic acid)
Nicotine salicylate (salicylic acid)
Nicotine 4-hydroxybenzoate (4-hydroxybenzoic acid)
Nicotine nicotinate (nicotinic acid)
Nicotine acetylsalicylate (acetylsalicylic acid)
Nicotine gallate (gallic acid)
Nicotine trimesate (trimesic acid)
Nicotine cinnamate (cinnamic acid)
Nicotine vanillate (vanillic acid)

### Example III

### Identification of Preferred Monocarboxylic Organic Acid-Nicotine Salt Complexes

It was determined, through experimentation, which of those embodiments were more preferable to others with respect to the monocarboxylic organic acid-nicotine salt complexes described in the present invention. Complexes were arranged in descending ranked order based on the following criteria:
(a) Auxiliary (free) functional groups: Molecules with the least amount of unbound carboxylate, alcohol or ester functional groups (other than those bound to nitrogens on the nicotine molecule) are the most preferred embodiments.
(b) Steric hindrance: An excess of molecular size is not preferable for embodiments described under this aromatic organic acid-nicotine salt complex.
(c) Possibility of forming higher order salts or bridges is preferred: An organic acid that possesses two or more carboxylic acids and or keto functional groups are preferred embodiments for the formation of a bridged complex if separated by 2-3 carbons between the carboxylic acid functional groups.

Further analysis revealed that nicotinic complexes with organic acids possessing a straight chain carbon tail would be the most preferred embodiment of this invention when classifying by the principle of free electron density. Nicotine benzoate would also be the most preferred embodiment when classifying by the principle of steric hinderance. Benzoate hydrogen bound to the pyrrolidine nitrogen would allow for the most relaxed bond angles when binding secondary organic acids to the pyridinyl nitrogen. For the same reason, nicotine benzoate would be the most preferred embodiment regarding the principle of higher order complexes.

Table 10 lists the most preferred monocarboxylic organic acid-nicotine salt complexes in order from most preferred to least preferred.

TABLE 10
Preferred Monocarboxylic Organic Acid-Nicotine Salt Complexes

Nicotine Formate (Formic Acid)
Nicotine Acetate (Acetic Acid)
Nicotine propiolate (propiolic acid)
Nicotine propiolate (propiolic acid)
Nicotine Butyrate (Butyric Acid)
Nicotine valerate (valeric acid)
Nicotine hexanoate (hexanoic acid)
Nicotine gluconate (gluconic acid)
Nicotine isocaproate (isocaproic acid)
Nicotine 2-hydroxyisocaproate (2-hydroxyisocaproic acid)
Nicotine benzoate (benzoic acid)
Nicotine salicylate (salicylic acid)
Nicotine 4-hydroxybenzoate (4-hydroxybenzoic acid)
Nicotine nicotinate (nicotinic acid)
Nicotine acetylsalicylate (acetylsalicylic acid)
Nicotine gallate (gallic acid)
Nicotine cinnamate (cinnamic acid)
Nicotine vanillate (vanillic acid)
Nicotine trimesate (trimesic acid)
Nicotine glycinate (glycine)
Nicotine alinate (alanine)
Nicotine serinate (serine)
Nicotine threoninate (threonine)
Nicotine cysteinate (cysteine)
Nicotine valinate (valine)
Nicotine leucinate (leucine)
Nicotine isoleucinate (isoleucine)
Nicotine methioninate (methionine)
Nicotine prolinate (proline)
Nicotine phenylanalinate (phenylalanine)
Nicotine tyrosinate (tyrosine)
Nicotine tryptophanate (tryptophan)
Nicotine asparaginate (asparagine)
Nicotine histidinate (histidine)
Nicotine lysinate (lysine)
Nicotine arginate (arginine)

### Example IV

### Identification of Preferred Dicarboxylic Organic Acid-Nicotine Salt Complexes

It was determined, through experimentation, which of those embodiments were more preferable to others with respect to the dicarboxylic organic acid-nicotine salt complexes described in the present invention. Complexes were arranged in descending ranked order based on the following criteria:
(a) Auxiliary (free) functional groups: Molecules with the least amount of unbound carboxylate, alcohol or ester functional groups (other than those bound to nitrogens on the nicotine molecule) are the most preferred embodiments.
(b) Steric hindrance: An excess of molecular size is not preferable for embodiments described under this aromatic organic acid-nicotine salt complex.
(c) Possibility of forming higher order salts or bridges is preferred: An organic acid that possesses two or more carboxylic acids and or keto functional groups are preferred embodiments for the formation of a bridged complex if separated by 2-3 carbons between the carboxylic acid functional groups.

Further analysis revealed that nicotine glutarate and nicotine succinate are ideal dicarboxylic acid choices due to their absence of excess chemical functional groups. The least sterically hindered choice on this list would be nicotine malonate or nicotine oxalate, possessing either 0 or 1 carbon separation, while dicarboxylic acids with between 2-4 carbons in length between the carboxylate functional groups, can bind to both the pyrollidinyl and pyridinyl nitrogens through hydrogen bonding. Nicotine glutarate and nicotine succinate are preferred embodiments of dicarboxylic acids eligible for bridging.

Table 11 lists the most preferred dicarboxylic organic acid-nicotine salt complexes in order from most preferred to least preferred.

TABLE 11
Preferred Dicarboxylic Organic Acid-Nicotine Salt Complexes

Nicotine oxalate (oxalic acid)
Nicotine malonate (malonic acid)
Nicotine acetoacetate (acetoacetic acid)
Nicotine tartrate (tartaric acid)
Nicotine succinate (succinic acid)
Nicotine fumarate (fumaric acid)
Nicotine trimesate (trimesic acid)
Nicotine adipate (adipic acid)
Nicotine aspartate (aspartic acid)
Nicotine glutamate (glutamic acid)
Nicotine acetylsalicylate (acetylsalicylic acid)

### Example V

### Identification of Preferred Tricarboxylic Organic Acid-Nicotine Salt Complexes

It was determined, through experimentation, which of those embodiments were more preferable to others with respect to the tricarboxylic organic acid-nicotine salt complexes described in the present invention. Complexes were arranged in descending ranked order based on the following criteria:
(a) Auxiliary (free) functional groups: Molecules with the least amount of unbound carboxylate, alcohol or ester functional groups (other than those bound to nitrogens on the nicotine molecule) are the most preferred embodiments.
(b) Steric hindrance: An excess of molecular size is not preferable for embodiments described under this aromatic organic acid-nicotine salt complex.
(c) Possibility of forming higher order salts or bridges is preferred: An organic acid that possesses two or more carboxylic acids and or keto functional groups are preferred embodiments for the formation of a bridged complex if separated by 2-3 carbons between the carboxylic acid functional groups.

Further analysis revealed that nicotine citrate and nicotine aconitate are ideal tricarboxylic acid choices due to their absence of excess chemical functional groups in comparison to trimezate, with excess electron density at the benzene central ring, which would be less preferred. The least sterically hindered choice on this list would be nicotine citrate or nicotine cis/trans aconitate in comparison to trimesate, with excess electron density at the benzene central ring, which would be less preferred. Tricarboxylic acids with between 2-4 carbons in length between the carboxylate functional groups, can bind to both the pyrollidinyl and pyridinyl nitrogens through hydrogen bonding. Nicotine dicitrate or nicotine N-citrate-N′-malateis are preferred embodiments of a tricarboxylic acid eligible for higher order binding into a homo or heterogeneous complex.

Table 12 lists the most preferred tricarboxylic organic acid-nicotine salt complexes in order from most preferred to least preferred.

TABLE 12
Preferred Tricarboxylic Organic Acid-Nicotine Salt Complexes

Nicotine citrate (citric acid)
Nicotine isocitrate (isocitric acid)
Nicotine cis-aconitate (cis-aconitic acid)
Nicotine trans-aconitate (trans-aconitic acid)
Nicotine trimesate (trimesic acid)

### Example VI

### Identification of Preferred Amino Acid-Nicotine Salt Complexes

It was determined, through experimentation, which of those embodiments were more preferable to others with respect to the amino acid-nicotine salt complexes described in the present invention. Complexes were arranged in descending ranked order based on the following criteria:
(a) Auxiliary (free) functional groups: Molecules with the least amount of unbound carboxylate, alcohol or ester functional groups (other than those bound to nitrogens on the nicotine molecule) are the most preferred embodiments.
(b) Steric hindrance: An excess of molecular size is not preferable for embodiments described under this aromatic organic acid-nicotine salt complex.
(c) Possibility of forming higher order salts or bridges is preferred: An organic acid that possesses two or more carboxylic acids and or keto functional groups are preferred embodiments for the formation of a bridged complex if separated by 2-3 carbons between the carboxylic acid functional groups.
(d) Overall charge of amino acid side chain: A non-charged or weekly charged amino acid is a more preferred embodiment than one that is strongly charged.

Further analysis revealed that glycine would be the most preferred embodiment when considering the lack of free functional groups as the principle for classification. Only the amino functional group is free on the glycine molecule when hydrogen is bound at the carboxylic acid functional group to a nitrogen on the nicotine molecule. When classifying based upon this principle, other amino acids with no other functional groups are also preferred embodiments, such as alanine, leucine, isoleucine, or valine. Nicotine glycinate would also be the most preferred embodiment when classifying by the principle of steric hinderance. Glycine hydrogen bound to the pyrrolidine nitrogen would allow for the most relaxed bond angles when binding secondary organic acids to the pyridinyl nitrogen. For the same reason, nicotine glycinate would be the most preferred embodiment regarding the principle of higher order complexes. Furthermore, glycine would be a more preferred embodiment compared to histidine, due to the fact that the glycine is an uncharged amino acid, whereas arginine possesses a strong positive charge.

Table 13 lists the most preferred amino acid-nicotine salt complexes in order from most preferred to least preferred.

TABLE 13
Preferred Amino Acid-Nicotine Salt Complexes

Nicotine glycinate (glycine)
Nicotine alinate (alanine)
Nicotine serinate (serine)
Nicotine threoninate (threonine)
Nicotine cysteinate (cysteine)
Nicotine valinate (valine)
Nicotine leucinate (leucine)
Nicotine isoleucinate (isoleucine)
Nicotine methioninate (methionine)
Nicotine prolinate (proline)
Nicotine phenylanalinate (phenylalanine)
Nicotine tyrosinate (tyrosine)
Nicotine tryptophanate (tryptophan)
Nicotine aspartate (aspartic acid)
Nicotine glutamate (glutamic acid)
Nicotine asparaginate (asparagine)
Nicotine histidinate (histidine)
Nicotine lysinate (lysine)
Nicotine arginate (arginine)

### Example VII

### Identification of Preferred Organic Acid-Nicotine Salt Complexes for Bridging (1:1 Bridged)

It was determined, through experimentation, which of those embodiments were more preferable to others with respect to the organic acid-nicotine salt complexes for bridging (1:1 bridged) described in the present invention. Complexes were arranged in descending ranked order based on the following criteria:
(a) Steric hindrance: An excess of molecular size is not preferable for embodiments described under this aromatic organic acid-nicotine salt complex.
(b) Possibility of forming higher order salts or bridges is preferred: An organic acid that possesses two or more carboxylic acids and or keto functional groups are preferred embodiments for the formation of a bridged complex if separated by 2-3 carbons between the carboxylic acid functional groups.

Further analysis revealed that nicotine malate 1:1 bridged is a preferred embodiment of the bridging principle due to the absence of electrical interference for hydrogen bonding at the two nitrogenous centers. Nicotine malate and succinate are preferred embodiments according to this principle, as the malate possesses 2 carbons while succinate possesses 3 carbons between the carboxylic acid moieties.

Table 14 lists the most preferred organic acid-nicotine salt complexes for bridging in order from most preferred to least preferred.

TABLE 14
Preferred Organic Acid-Nicotine Salt
Complexes for Bridging (1:1 Bridged)

Nicotine tartrate (tartaric acid)
Nicotine malate (malic acid)
Nicotine glutarate (glutaric acid)
Nicotine glutamate (glutamic acid)
Nicotine acetylsalicylate (acetylsalicylic acid)
Nicotine succinate (succinic acid)
Nicotine aspartate (aspartic acid)
Nicotine glutamate (glutamic acid)
Nicotine trimesate (trimesic acid)

### Example VIII

### Identification of Preferred Electron-Poor Organic Acids Yielding a More pH Balanced (or “Smoother”) Nicotine-Salt Complex Character when Used Orally, Intranasally or via Intrapulmonary Administration

It was determined, through experimentation, which of those embodiments were more preferable to others with respect to the electron-poor organic acids yielding a more balanced pH nicotine-salt complex described in the present invention. Complexes were arranged in descending ranked order based on the following criteria:
(a) Auxiliary (free) functional groups: Molecules with the least amount of unbound carboxylate, alcohol or ester functional groups (other than those bound to nitrogens on the nicotine molecule) are the most preferred embodiments. A keto functional group is the most preferred unbound functional group.
(b) Molar ratio: Using organic acids with the above properties in a high molar ratio (preferably 2:1) is most preferred, as bonding with the pyridinyl nitrogen contributes to the smooth character.

Further analysis revealed that a preferred example of a smooth complex would be “bridged” 1:1 nicotine malate. Both carboxylic acid functional groups are bound to both nitrogens on the nicotine molecule, forming a bridge, leaving no exposed functional groups on the malate molecule. Another example is nicotine levulinate, which only has a keto functional group and has the potential to bridge if the pH of the mixture is above the pKa 4.64.

Table 15 lists the most preferred electron-poor organic acids yielding a more balanced pH nicotine-salt complex character, ranked in order from most preferred to least preferred.

TABLE 15
Preferred Electron-Poor Organic Acids Yielding a More Balanced
pH Nicotine-Salt Complex for the “Smooth” Character

Nicotine levulinate (levuliniuc acid)
Nicotine pyruvate (pyruvic acid)
Nicotine hydroxypyruvate (hydroxypyruvic acid)
Nicotine lactate (lactic acid)
Nicotine Acetate (acetic acid)
Nicotine formate (formic acid)
Nicotine propionate (propionic acid)
Nicotine propiolate (propiolic acid)
Nicotine Butyrate (butyric acid)
Nicotine valerate (valeric acid)
Nicotine isovalerate (isovaleric acid)
Nicotine hexanoate (hexanoic acid)
Nicotine isocaproate (isocaproic acid)
Nicotine 2-hydroxyisocaproate (2-hydroxyisocaproic acid)
Nicotine glutarate (glutaric acid)
Nicotine 3-hydroxyglutarate (3-hydroxyglutaric acid)
Nicotine adipitate (adipitic acid)
Nicotine glycinate (glycine)
Nicotine alinate (alanine)
Nicotine serinate (serine)
Nicotine threoninate (threonine)
Nicotine cysteinate (cysteine)
Nicotine valinate (valine)
Nicotine leucinate (leucine)
Nicotine isoleucinate (isoleucine)
Nicotine methioninate (methionine)
Nicotine prolinate (proline)
Nicotine phenylanalinate (phenylalanine)
Nicotine tyrosinate (tyrosine)
Nicotine tryptophanate (tryptophan)
Nicotine asparaginate (asparagine)
Nicotine histidinate (histidine)

### Example IX

### Identification of Preferred Electron-Rich Organic Acids Yielding a Lower pH (“Throat Hit”) or a Positively Associated, Irritant-Inducing Nicotine-Salt Complex Character when Used Orally, Intranasally or via Intrapulmonary Administration

It was determined, through experimentation, which of those embodiments were more preferable to others with respect to the electron-rich organic acids yielding a lower pH nicotine-salt complex described in the present invention. Complexes were arranged in descending ranked order based on the following criteria:
(a) Auxiliary (free) functional groups: Molecules with two or more carboxylate, alcohol or ester, and aromatic functional groups, other than those bound to the nitrogens on the nicotine molecule, are the most preferred embodiments.
(b) Electron density: Functional groups are preferred when they provide high electron density, for example, free carboxylic acid groups and aromatic rings.

Further analysis revealed that a preferred example of a complex exhibiting a “throat hit” character would be nicotine N′-oxalate-N-acetoacetate. Two carboxylic acid functional groups, one on the oxalic acid and the other on the acetoacetic acid molecules, would contribute to a strong throat hit. The functional groups do not have to have acidic character to allow for “throat hit”. A molecule like nicotine dibenzoate, which possesses two free benzene moieties left unbound to the nitrogens on the nicotine molecule, possesses a great amount of pharengeal irritation in the form of a “throat hit” due to the high electron density of the aromatic rings.

Table 16 lists the most preferred electron-rich organic acids yielding a lower pH nicotine-salt complex, ranked in order from most preferred to least preferred.

TABLE 16
Preferred Electron-Rich Organic Acids Yielding a Lower pH Nicotine-
Salt Complex Resulting in a “Throat Hit” Character

Nicotine 4-hydroxybenxoate (4-hydroxybenxoic acid)
Nicotine oxalate (oxalic acid)
Nicotine malonate (malonic acid)
Nicotine gluconate (gluconic acid)
Nicotine tartrate (tartaric acid)
Nicotine malate (malic acid)
Nicotine succinate (succinic acid)
Nicotine oxaloacetate (oxaloacetic acid)
Nicotine acetoacetate (acetoacetic acid)
Nicotine fumarate (fumaric acid)
Nicotine benzoate (benzoic acid)
Nicotine salicylate (salicylic acid)
Nicotine acetylsalisylate (acetylsalisylic acid)
Nicotine phthalate (phthalic acid)
Nicotine cinnamate (cinnamic acid)
Nicotine gallate (gallic acid)
Nicotine trimesate (trimesic acid)
Nicotine cis-aconitate (cis-aconitic acid)
Nicotine trans-aconitate (trans-aconitic acid)
Nicotine nicotinate (nicotinic acid)
Nicotine citrate (citric acid)
Nicotine isocitrate (isocitric acid)
Nicotine vanillate (vanillic acid)
Nicotine glutamate (glutamic acid)
Nicotine aspartate (aspartic acid)
Nicotine lysinate (lysine)
Nicotine arginate (arginine)
Nicotine 3-mercaptopyruvate (3-mercaptopyruvic acid)
Nicotine beta-ketoglutarate (beta-ketoglutaric acid)
Nicotine alpha-ketoglutarate (alpha-ketoglutaric acid)
Nicotine 4-hydroxy-2-oxopentanoate (4-hydroxy-2-oxopentanoic acid)
Nicotine 4-hydroxy phenylpyruvate (4-hydroxy phenylpyruvic acid)

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features. As used in this specification and in the appended claims, the singular forms include the plural forms. For example the terms “a,” “an,” and “the” include plural references unless the content clearly dictates otherwise. Additionally, the term “at least” preceding a series of elements is to be understood as referring to every element in the series. The inventions illustratively described herein can suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the future shown and described or any portion thereof, and it is recognized that various modifications are possible within the scope of the invention claimed. Thus, it should be understood that although the present invention has been specifically disclosed by preferred embodiments and optional features, modification and variation of the inventions herein disclosed can be resorted by those skilled in the art, and that such modifications and variations are considered to be within the scope of the inventions disclosed herein. The inventions have been described broadly and generically herein. Each of the narrower species and subgeneric groupings falling within the scope of the generic disclosure also form part of these inventions. This includes the generic description of each invention with a proviso or negative limitation removing any subject matter from the genus, regardless of whether or not the excised materials specifically resided therein. In addition, where features or aspects of an invention are described in terms of the Markush group, those schooled in the art will recognize that the invention is also thereby described in terms of any individual member or subgroup of members of the Markush group. It is also to be understood that the above description is intended to be illustrative and not restrictive. Many embodiments will be apparent to those of in the art upon reviewing the above description. The scope of the invention should, therefore, be determined not with reference to the above description, but should instead be determined with reference to the appended claims, along with the full scope of equivalents to which such claims are entitled. Those skilled in the art will recognize, or will be able to ascertain using no more than routine experimentation, many equivalents to the specific embodiments of the invention described. Such equivalents are intended to be encompassed by the following claims.

What is claimed is:

1. A solution, comprising: one or more heterogenous nicotine salt complexes, wherein the complexes comprise at least one nicotine molecule having a first organic acid bound to a nitrogen of a pyrrolidine ring and a second organic acid bound to a nitrogen of a pyridine ring.

2. The solution of claim 1, wherein the first organic acid and second organic acid are selected from the group consisting of: 2-hydroxyisocaproic acid, 3-hydroxyglutaric acid, 4-hydroxybenzoic acid, acetic acid, acetoacetic acid, acetylsalicylic acid, adipic acid, alanine, arginine, asparagine, aspartic acid, benzoic acid, cinnamic acid, cis-aconitic acid, citric acid, cysteine, formic acid, fumaric acid, gallic acid, gluconic acid, glutamic acid, glutaric acid, glycine, glycolic acid, hexanoic acid, histidine, isocaproic acid, isocitric acid, isoleucine, isovaleric acid, lactic acid, leucine, levuliniuc acid, lysine, malic acid, malonic acid, methionine, nicotinic acid, oxalic acid, phenylalanine, phthalic acid, proline, propiolic acid, propionic acid, pyruvic acid, salicylic acid, serine, succinic acid, tartaric acid, threonine, trans-aconitic acid, trimesic acid, tryptophan, tyrosine, valeric acid, valine, and vanillic acid.

3. The solution of claim 1, including three or more organic acids forming heterogenous nicotine salt complexes.

4. The solution of claim 1, including four or more organic acids forming heterogenous nicotine salt complexes.

5. The solution of claim 1, having a pH from 3.0 to 8.0.

6. The solution of claim 1, having a pH from 5.0 to 7.0.

7. The solution of claim 1, having a pH from 5.0 to 6.5.

8. The solution of claim 1, wherein the solution contains the compound nicotine and the nicotine concentration is from 50 mg/ml to 750 mg/ml.

9. The solution of claim 1, wherein the solution contains the compound nicotine and the nicotine concentration is from 100 mg/ml to 750 mg/ml.

10. The solution of claim 1, wherein the first organic acid is selected from the group consisting of: a monocarboxylic organic acid, a dicarboxylic organic acid, a tricarboxylic organic acid and an aromatic organic acid.

11. The solution of claim 1, wherein a first organic acid and a second organic acid are selected column A and column B, respectively, in Table 1, copied in the present claim.

12. The solution of claim 1, further comprising, one or more nicotine salts selected for a smooth vaping experience for use a delivery mode selected from the group consisting essentially of: a transdermal device, an oral delivery device, an intranasal delivery device and a respiratory delivery device.

13. The solution of claim 1, further comprising: one or more nicotine salts selected for a biting experience and packaged for a delivery mode selected from the group consisting essentially of: an oral and a respiratory delivery device.

14. The solution of claim 1, further comprising, a packaging selected for transdermal, oral, nasal, or respiratory delivery.

15. A solution, comprising: a nicotine salt complex including a conjugate base of an organic acid selected from the group consisting of: 2-hydroxyisocaproic acid, 3-hydroxyglutaric acid, 4-hydroxybenzoic acid, acetic acid, acetoacetic acid, acetylsalicylic acid, adipic acid, alanine, arginine, asparagine, aspartic acid, benzoic acid, cinnamic acid, cis-aconitic acid, citric acid, cysteine, formic acid, fumaric acid, gallic acid, gluconic acid, glutamic acid, glutaric acid, glycine, glycolic acid, hexanoic acid, histidine, isocaproic acid, isocitric acid, isoleucine, isovaleric acid, lactic acid, leucine, levuliniuc acid, lysine, malic acid, malonic acid, methionine, nicotinic acid, oxalic acid, phenylalanine, phthalic acid, proline, propiolic acid, propionic acid, pyruvic acid, salicylic acid, serine, succinic acid, tartaric acid, threonine, trans-aconitic acid, trimesic acid, tryptophan, tyrosine, valeric acid, valine, and vanillic acid.

16. The solution of claim 15, having a pH in a range from 3.0 to 8.0.

17. A solution, comprising: a nicotine salt complex selected from the group consisting of: nicotine glutarate, nicotine 3-hydroxyglutarate, nicotine leucinate, nicotine valinate, nicotine isoleucinate, nicotine alinate, nicotine arginate, nicotine lysinate, nicotine glutamate, nicotine aspartate, nicotine prolinate, nicotine cysteinate, nicotine threoninate, nicotine methioninate, nicotine histidinate, nicotine phenylanalinate, nicotine tyrosinate, nicotine tryptophanate, nicotine asparaginate, nicotine glycinate, and nicotine serinate.

18. The solution of claim 17, having a pH of from 3.0 to 8.0.

19. The solution of claim 17, having a pH of from 4.9 to 6.1.

20. The solution of claim 17, wherein a pH value of the solution is between 4.0 and 6.5.

21. The solution of claim 17, wherein the complex includes a nicotine compound having a concentration from 50 mg/ml to 750 mg/ml.

22. A nicotine containing solution, comprising: one or more nicotine salt complexes suitable for a delivery mode selected from the group consisting of: transdermal, oral, inhalation, insufflation, catheterization, and injection.

23. The nicotine containing solution of claim 22, having a reduced nicotine bite.

24. An oral nicotine delivery aid, comprising: a nicotine salt complex.

25. The oral nicotine delivery aid of claim 24, further comprising a lozenge or a gum including the nicotine salt complex.

26. The oral nicotine delivery aid of claim 24, further comprising a lozenge including the nicotine salt complex.

27. The oral nicotine delivery aid of claim 24, further comprising a chewing gum including the nicotine salt complex.

28. A nicotine salt complex, comprising: a nicotine molecule having a pyridine ring having a first nitrogen atom and a pyrrolidine ring having a second nitrogen atom and an organic moiety complexed with the first and the second nitrogen atoms, thereby forming a bridge.

29. A nicotine salt complex, comprising: a nicotine molecule and an organic acid having two or more carboxylic acid moieties, separated by greater than 1 but no more than 3 carbons, wherein at least a first and a second carboxylic acid moiety are hydrogen bound to a pyrrolidinyl nitrogen and a pyridinyl nitrogen of the nicotine molecule.

30. The nicotine salt complex of claim 29, wherein the organic acid forms a bridge between a first and a second nitrogen centers of a nicotine molecule.

31. A method for formulating or manufacturing a nicotine containing solution for a selected user experience of bite, smooth or an experience therebetween, comprising: selecting one or more organic acids based on one or more factors selected from a group consisting essentially of: a pKa value, a final pH, an electronegativity, a functional group other than a first carboxylic functional group, a molecular weight, a molecular dimension, and a branching carbon structure; and specifying or combining the nicotine and the one or more organic acids thereby formulating or manufacturing the solution.

32. The method of claim 31, wherein the solution is combined with a device for a nicotine delivery mode of: transdermal, oral, inhalation, insufflation, catheterization, and injection.

33. A method for formulating or manufacturing a nicotine containing solution for a selected user experience of bite, smooth or an experience therebetween, comprising: selecting one or more pre-made solutions, each solution having an indicator therewith setting forth an impact of the solution on the experience and specifying or combining the selected pre-made solutions.

34. The method of claim 33, wherein the solution is combined with a device for a nicotine delivery mode of: transdermal, oral, inhalation, insufflation, catheterization, and injection.

35. A kit for formulating or manufacturing a user or a practitioner determined use experience of a nicotine containing solution, comprising: a nicotine solution and one or more organic acid solutions, a nicotine solution and one or more pre-made nicotine salt solutions, or two or more nicotine salt solutions.

36. The kit of claim 35, containing the one or more organic acid solutions, wherein the organic acids based on one or more factors selected from a group consisting essentially of: a pKa value, a final pH, an electronegativity, a functional group other than a first carboxylic functional group, a molecular weight, a molecular dimension, and a branching carbon structure; and specifying or combining the nicotine and the one or more organic acids thereby formulating or manufacturing the solution.

37. A method for manufacturing a solution including a heterogenous nicotine salt complex in a solution, comprising: providing a nicotine compound and at least two organic acids, wherein at least one organic acid having a profile and admixing the nicotine compound and the organic acids.

38. A composition comprising one or more nicotine salt in a solution for vaping, comprising: a nicotine molecule and from zero to one or more dicarboxylic acids and one or more keto acids, wherein the nicotine molecule and the acid form the one or more nicotine salts, wherein the solution has a pH above 6.7.

39. A composition comprising three or more nicotine salts in a solution for vaping, comprising: one or more nicotine monocarboxylic acids, one or more nicotine dicarboxylic acids and one or more nicotine keto acids forming the one or more nicotine salts.
